(19) United States
(12) Patent Application Publication
(10) Pub. No.: US 2006/0041956 A1
(43) Pub. Date: Feb. 23, 2006

(54) Vectors for plant transformation and methods of use

(75) Inventors: Michael Lassner, Foster City, CA (US); Kevin E. McBride, Davis, CA (US); Jack Q. Wilkinson, Redwood City, CA (US); Sean M. Bertain, Piedmont, CA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: Pioneer Hi-Bred International, Inc.

(21) Appl. No.: 11/100,258

(22) Filed: Apr. 6, 2005

Related U.S. Application Data
(60) Provisional application No. 60/559,895, filed on Apr. 6, 2004.

(51) Int. Cl. C12N 15/82; A01H 1/00; C12N 15/87

(52) U.S. Cl. 800/282; 800/294

(57) ABSTRACT

The present invention is directed to a vector for identifying read-through of non-T-DNA in a T-DNA vector. In one embodiment, the vector provides a visually detectable change in the normal appearance of transformants wherein read-through has occurred. In another embodiment, the vector also provides for expression of a readily detectable fluorescent protein that allows for the early detection and elimination of transformants wherein read-through has occurred. In a further aspect, the present invention is directed to a method for detecting read-through of non-T-DNA in plants transformed with a T-DNA vector. In another aspect, the present invention is directed to a method for producing a transgenic plant containing a polynucleotide of interest but being substantially free of non-T-DNA.

### CROSS REFERENCE TO RELATED APPLICATIONS

This application claims priority to U.S. Ser. No. 60/559,895 filed Apr. 6, 2004 the disclosure of which is incorporated herein by reference.

### FIELD OF THE INVENTION

The present invention relates to a plant transformation vector for use with Agrobacterium species (A. tumefaciens, A. rhizogenes).

### BACKGROUND OF THE INVENTION

Naturally occurring A. tumefaciens forms a tumorous outgrowth, “crown gall,” on infected dicotyledonous plants (dicots). The related bacterium, A. rhizogenes, forms a tumorous outgrowth, “hairy roots,” on infected dicots. The molecular basis for this tumorous growth is the transfer of certain genetic material from the bacterium to the genome of the infected plant. The transferred DNA, also called “T-DNA,” contains the bacterial genes that are expressed in the infected plant cells. These transferred bacterial genes encode proteins involved in hormone biosynthesis or action, as well as proteins involved in producing bacterial metabolites. The expression of these bacterial genes deregulates the plant's normal controls for cell division and results in the uncontrolled tumorous growth of the bacterially infected cells.

Although the natural targets of A. tumefaciens or A. rhizogenes are dicots, they are also used to transform yeast, molds and filamentous fungi. See Bundock et al., “Trans-kingdom T-DNA transfer from Agrobacterium tumefaciens to Saccharomyces cerevisiae,” EMBO J., 14: 3206-3214 (1995); de Groot et al., “Agrobacterium tumefaciens-mediated transformation of filamentous fungi,” Nat. Biotechnol., 16:839-842 (1998); Gouka et al., “Transformation of Aspergillus awamori by Agrobacterium tumefaciens-mediated homologous recombination,” Nat. Biotechnol., 17, 598-601 (1999); and WO98/45455. More recently, Agrobacterium has been utilized to transform monocot plant species such as rice and maize. See Hiei et al., The Plant Journal 6: 271-282 (1994); U.S. Pat. No. 5,591,616, issued Jan. 7, 1997, all of which are incorporated herein by reference. In addition, components of the T-DNA production and transfer machinery of Agrobacterium are useful for importing DNA into the nuclei of mammalian cells, opening perspectives for use of these components in gene therapy. See Ziemienowicz et al., “Import of DNA into mammalian nuclei by proteins originating from a plant pathogenic bacterium,” Proc. Natl. Acad. Sci. U.S.A 96, 3729-3733 (1999). Thus, Agrobacterium can be used to transfer any DNA located on the T-DNA into the nucleus of a eukaryotic cell. Other non-Agrobacterium species have also been utilized in transformation (Nature 433(7026):629-633 2005).

In A. tumefaciens, the T-DNA is part of the wild-type Ti-plasmid; in A. rhizogenes, the T-DNA is part of the wild-type Ri-plasmid. In the case of plants, the Ti plasmids have the unique capability to transform the cells of susceptible plants by inserting an 8 to 32 kilobase segment of T-DNA into the host plant's chromosomal DNA. Transformed plant cells containing wild-type T-DNA grow in culture without an exogenous supply of auxin or cytokinin. In contrast, normal plant cells require both substances for growth in culture. Mutations in one T-DNA locus cause tumors from which abundant roots proliferate (“rooty” mutants), while mutations in a second T-DNA locus cause tumors from which shoots proliferate (“shooty” mutants). See Ooms et al., Gene, 14:33-50 (1981); and Garfinkel et al., Cell, 27:143-153 (1981).

Wild-type Ti- or Ri-plasmids also carry vir genes (virulence genes) which are activated by plant phenolic compounds. The expression products of the vir genes are responsible for the transfer of the T-DNA into the eukaryotic genome. For transformation purposes, the T-DNA is disarmed (i.e., all oncogenic and disease-causing genes are removed) and vir genes are supplied either in trans or on a helper plasmid. The T-DNA encompassing heterologous gene(s) is then located on a second binary plant transformation vector or in cis in case of a co-integrate plant transformation vector. The heterologous genes of interest are cloned into the T-DNA region, which is situated between the two T-DNA 22 bp (in the case of octopine Ti plasmids) or 25 bp (in the case of nopaline Ti plasmids) imperfect border core sequences constituting the right border (RB) and the left border (LB). The border core sequences of the RB and LB, which are the only in cis elements necessary to direct T-DNA processing, are organized as imperfect repeats.

For many years it was believed that only the T-DNA between the RB and LB repeats was transferred to the target plant cell. However, recent and more detailed characterization of the DNA inserts in transgenic plants demonstrates that vector backbone sequences (outside the T-DNA region) integrate very frequently into the plant's genome. See Ramanathan and Veluthambi, “Transfer of non-T-DNA portions of the Agrobacterium tumefaciens Ti plasmid pTiA6 from the left terminus of T.sub.L-DNA,” Plant Mol. Biol., 28: 1149-1154 (1995); Cluster et al., “Details of T-DNA structural organization from a transgenic Petunia population exhibiting co-suppression,” Plant Mol. Biol. 32: 1197-1203 (1996); van der Graaff et al., “Deviating T-DNA transfer from Agrobacterium tumefaciens to plants,” Plant Mol. Biol. 31: 677-681 (1996); Martineau et al., “On defining T-DNA,” Plant Cell 6: 1032-1033 (Letter to the editor) (1994); Wenck et al., “Frequent collinear long transfer of DNA inclusive of the whole binary vector during Agrobacterium-mediate transformation,” Plant Mol. Biol. 34: 913-922 (1997); and Wolters et al., “Fluorescence in situ hybridization on extended DNA fibers as a tool to analyze complex T-DNA loci in potato,” Plant J., 13: 837-847 (1998).

One problem with “beyond the border” DNA transfer is the potential expression in a transformed plant of undesired foreign proteins encoded by the “beyond the border” DNA. These foreign proteins, even if totally harmless, are considered to pose an added and unnecessary risk by a significantly large portion of the population who would refuse to buy any transformed plant expressing such undesired foreign proteins, thus making the transformed plant economically undesirable. One of the reasons given by the public for undesirably of such a plant is the contention that such a foreign protein may be antigenic and could give rise to a food allergy in a sensitized individual. Moreover, market approval by government agencies for transformed plants expressing proteins encoded by “beyond the border” DNA would be more costly, require more testing and provide less certainty of eventual approval.

The frequency of “beyond the border” DNA transfers has been reported to vary from 5% to over 75%. See Kononov et al., “Integration of T-DNA binary vector ‘backbone’ sequences into the tobacco genome: evidence for multiple complex patterns of integration,” Plant J. 11: 945-957 (1997). Thus, it is an object of the present invention to provide a transformation vector that is more effective than the wild-type Ti-plasmid in minimizing the transfer of “beyond the border” DNA to a transformed organism or plant. More specifically, the vector of the present invention will allow the identification and elimination of transformed cells, organisms, and plants in which unwanted DNA transfer has occurred.

WO 99/01563 (Stuiver et al.) discloses one approach to minimizing or preventing read-through at the borders. Specifically, Stuiver et al. discloses the creation of DNA binding sites outside the T-borders to prevent read-through. In addition, Stuiver et al. discloses the insertion of coding sequences outside the T-borders that are toxic to the plants to provide counterselection for plants with the superfluous vector-DNA.

Another approach to minimizing or preventing read-through at the borders is disclosed in U.S. App 2003/0140376 (Depicker et al.) which published on Jul. 24, 2003. The approach of Depicker et al. was multifaceted and generally involved modifying the left border region to increase the efficient processing of the left border by the nicking complex involving VirD1 and VirD2, or allowing for the excision of vector backbone sequences.

A third approach to preventing read-through in a T-DNA vector is disclosed in EP 1 136 560 A1. In EP 1 136 560 A1, the applicants modified the left border so that it is more recognizable by the vir proteins. In a preferred embodiment, the left border was modified to include a plurality of left borders, typically, 2 to 6 left border sequences, more typically, 2 to 5 left border sequences, most typically 2 to 4 left border sequences.

Another approach to addressing beyond the border read-through is to provide for the negative selection of those plants in which read-through has occurred. U.S. Pat. No. 6,521,458, entitled “Compositions and Methods for Improved Plant Transformations,” issued on Feb. 18, 2003. In this patent, Gutterson et al. disclose a method for eliminating plants containing non-T-DNA sequence derived from a T-DNA vector by incorporating a lethal polynucleotide sequence outside the left border region. Any read-through beyond the left border would allow for selection of plants that do not contain the lethality gene.

An entirely different approach to making T-DNA vectors more useful is disclosed in U.S. Pat. No. 6,051,757, entitled “Regeneration of plants containing genetically engineered T-DNA,” which issued to Barton et al. on Apr. 18, 2002. In this patent, Barton et al. disclose that it is advantageous to disarm the cytokinin autonomy gene, which is normally situated in the T-DNA region of a wild-type Ti-plasmid (T-DNA vector). According to Barton et al., the elimination of the cytokinin autonomy gene or its function (i.e., by “disarming” the T-DNA), such that the entire T-DNA vector lacks a functional cytokinin autonomy gene, permits the regeneration of complete plants with roots containing one or more full length copies of the genetically engineered T-DNA.

Because the frequency of “beyond the border” DNA transfer from a wild-type Ti plasmid is at least 5% and as high as 75%, it would be very useful to provide a Ti-based plant transformation vector having a marker gene that would provide for the efficient screening of those transformants that contain the expression of proteins encoded by “beyond the border” DNA (i.e., DNA outside the T-DNA region).

### SUMMARY OF THE INVENTION

The present invention relates to a plant transformation vector for use with Agrobacterium species (A. tumefaciens, A. rhizogenes) or other bacterial species (Sinorhizobium, Rhizobium, and Mesorhizobium) that can be utilized to produce transgenic plants. More particularly, the present invention relates to a plant transformation vector, i.e., an engineered Ti plasmid, that contains a visual reporter gene to facilitate the elimination of transformants that are transformed with DNA from beyond the T-DNA region of the vector. The present invention is useful for transforming plants, particularly those intended for consumption by humans and/or animals where the presence of extraneous genetic material would pose an added and unnecessary risk.

### DETAILED DESCRIPTION OF THE INVENTION

The present invention is directed to an isolated T-DNA vector for identifying read-through of non-T-DNA in Agrobacterium-mediated plant transformation. In one embodiment, the T-DNA vector comprises: i) a right border, a left border and a T-DNA sequence therebetween, the T-DNA sequence therebetween lacking a functional cytokinin autonomy gene; and ii) a non-T-DNA sequence beyond the left border, the non-T-DNA sequence comprising an Agrobacterium origin of replication and a first visual marker gene, the first visual marker gene being a cytokinin autonomy gene. Any read-through of the cytokinin autonomy gene in the non-T-DNA region may produce visually discernable changes in the transformed material such as abnormal cell proliferation, accelerated or deeper “greening,” delayed senescence, the formation of “shooty” tumors, or regenerated plantlets having altered morphology due to cytokinin over-production (Taiz and Zeiger, Plant Physiology, E. Brady (ed.) The Benjamin/Cummings Publishing Company, Inc., 1991). In certain systems, the “shooty” appearance of the transformants may never be reached. The cells with the cytokinin autonomy gene may just proliferate in tissue culture and not become shoots or plantlets but are otherwise clearly identifiable as later disclosed herein.

Optionally, the non-T-DNA sequence further comprises an Agrobacterium vir gene, or a polynucleotide encoding one or more Agrobacterium vir proteins, typically 1 to 5 vir proteins, more typically, 1 to 3 vir proteins, most typically 1 to 2 vir proteins. Providing additional copies of certain vir genes or operons on the T-DNA vector may enhance T-strand formation and plant transformation efficiency. See Wang et al., J. Bacteriology, 172:4432-4440, 1990.

In another embodiment, the non-T-DNA sequence of the above-described vector also comprises a second visual marker gene, such as a gene encoding a visually detectable fluorescent protein. Any transformant having “read-through” that causes expression of the second visual marker gene will be visually detectable by illuminating the transformant with an excitation frequency for the fluorescent protein and checking the illuminated transformants for the appropriate color of fluorescence. The excitation and fluorescent wavelengths for various fluorescent proteins are disclosed in Table 1 herein. Thus, the vector of the present invention allows for the early detection and elimination of those transformants wherein read-through has occurred.

Alternatively, the second visual marker gene encodes a protein whose expression triggers the production of a visually detectable chemical compound. Examples of such genes include those involved in the regulation or biosynthesis of anthocyanin, carotenoid, or indigo pigments. See Walbot et al., Basic Life Sci. 41:183-188 (1987); Lloyd et al., Science, 258:1773-1775 (1992); Borevitz et al., Plant Cell, 12:2383-2394 (2000); Holton, Drug Metabol Drug Interact., 12:359-368 (1995); Sandmann, Arch Biochem Biophys., 385:4-12 (2001); Mann et al., Nat. Biotechnol. 18:888-892 (2000); and Minami et al., Plant Cell Physiol., 41:218-225 (2000). Any transformant having “read-through” that causes expression of these marker genes will be visually detectable by examining the transformant under visible light and checking for the expected color of the illuminated chemical compound(s). Thus, the vector of the present invention allows for the early detection and elimination of those transformants wherein read-through has occurred.

It is also within the scope of the T-DNA vector of the present invention that the left border be modified to comprise a plurality of left border sequences, typically 2 to 6 left border sequences, more typically, 2 to 5 left border sequences, most typically 2 to 4 left border sequences. Each left border sequence is recognizable by an Agrobacterium vir protein and is capable of being nicked to prevent read-through and non-T-DNA insertion into a host genome. The use of a plurality of left border sequences merely increases the likelihood of recognition of the left border by a vir protein and decreases the likelihood of read-through.

In a further embodiment, the present invention is directed to a method for detecting read-through of non-T-DNA in plants transformed with a T-DNA vector, the method comprising:
(a) transforming a plurality of plant cells with a T-DNA vector comprising:
   (i) a right border, a left border and a T-DNA sequence therebetween, the T-DNA sequence therebetween lacking a functional cytokinin autonomy gene; and
   (ii) a non-T-DNA sequence beyond the left border, the non-T-DNA sequence comprising an Agrobacterium origin of replication and a first visual marker gene, the first visual marker gene being a cytokinin autonomy gene; and
(b) detecting those transformed plant cells with one or more of the following: an altered phenotype, abnormal cell proliferation, accelerated or deeper “greening,” delayed senescence, or the formation of “shooty” tumors which are associated with read-through and expression of the cytokinin autonomy gene from the non-T-DNA sequence.

Optionally, the non-T-DNA sequence employed in the above-described method further comprises an Agrobacterium vir gene, or a polynucleotide encoding one or more Agrobacterium vir proteins, typically 1 to 5 vir proteins, more typically, 1 to 3 vir proteins, most typically 1 to 2 vir proteins. In another embodiment the cytokinin autonomy gene is from Agrobacterium tumefaciens. In another embodiment the Agrobacterium origin of replication is derived from the ori region of the Pseudomonas PVS1 plasmid or the ori region of the RK2 broad-host range plasmids. In another embodiment of the above method the left border has been modified to comprise more than one left border sequence. In another embodiment of the above method, the non-T DNA sequence further comprises a gene encoding a second visual marker, such as a gene encoding a fluorescent protein, or a gene encoding a protein involved in the regulation or biosynthesis of anthocyanin, carotenoid, or indigo pigments. Preferably, the second visual marker is a gene encoding a fluorescent protein, more preferably, a green fluorescent protein.

In another aspect, the present invention is directed to a method for producing a transgenic plant containing a polynucleotide of interest, the method comprising:
(a) transforming a plurality of plant cells with a T-DNA vector comprising:
   (i) a right border, a left border and a T-DNA sequence therebetween, the T-DNA sequence therebetween lacking a functional cytokinin autonomy gene; and
   (ii) a non-T-DNA sequence beyond the left border, the non-T-DNA sequence comprising an Agrobacterium origin of replication and a first visual marker gene, the first visual marker gene being a cytokinin autonomy gene;
(b) selecting a transformed plant cell and its progeny which integrate and express the T-DNA sequence and do not visually manifest abnormal cell proliferation, accelerated or deeper “greening,” delayed senescence, the formation of “shooty” tumors, or other phenotypes associated with read-through and expression of the cytokinin autonomy gene from the non-T-DNA sequence; and
(c) regenerating a transgenic plant from the selected plant cell.

Optionally, the non-T-DNA sequence employed in the above-described method further comprises an Agrobacterium vir gene, or a polynucleotide encoding one or more Agrobacterium vir proteins, typically 1 to 5 vir proteins, more typically, 1 to 3 vir proteins, most typically 1 to 2 vir proteins. Another embodiment of the above-described method the cytokinin autonomy gene in the non-T-DNA sequence is from Agrobacterium tumefaciens. In another embodiment, the Agrobacterium origin of replication is derived from the ori region of the Pseudomonas PVS1 plasmid or the ori region of the RK2 broad-host range plasmids. In another embodiment the left border has been modified to comprise from more than one left border sequence. In another embodiment of the above method, the non-T DNA sequence further comprises a gene encoding a second visual marker, such as a gene encoding a fluorescent protein, or a gene encoding a protein involved in the regulation or biosynthesis of anthocyanin, carotenoid, or indigo pigments. Preferably, the second visual marker is a gene encoding a fluorescent protein, more preferably, a green fluorescent protein.

In a further embodiment, the present invention is directed to a method for detecting read-through of non-T-DNA in plants transformed with a T-DNA vector, the method comprising:
(a) transforming a plurality of plant cells with a T-DNA vector comprising:
   (i) a right border, a left border and a T-DNA sequence therebetween, the T-DNA sequence therebetween lacking a functional cytokinin autonomy gene; and
   (ii) a non-T-DNA sequence beyond the left border, the non-T-DNA sequence comprising an Agrobacterium origin of replication, a first visual marker gene, and a second visual marker gene, wherein the first visual marker gene is a cytokinin autonomy gene and the second visual marker gene encodes a visually detectable fluorescent protein; and
(b) detecting those transformed plant cells with visible fluorescence upon illumination with an appropriate light source, wherein fluorescence is associated with read-through and expression of the fluorescent protein gene from the non-T-DNA sequence.

Optionally, the non-T-DNA sequence employed in the above-described method further comprises an Agrobacterium vir gene, or a polynucleotide encoding one or more Agrobacterium vir proteins, typically 1 to 5 vir proteins, more typically, 1 to 3 vir proteins, most typically 1 to 2 vir proteins.

In another embodiment, the present invention is directed to a method for producing a transgenic plant containing a polynucleotide of interest but being substantially free of non-T-DNA, the method comprising:
(a) transforming a plurality of plant cells with a T-DNA vector comprising:
   (i) a right border, a left border and a T-DNA sequence therebetween, the T-DNA sequence therebetween lacking a functional cytokinin autonomy gene; and
   (ii) a non-T-DNA sequence beyond the left border, the non-T-DNA sequence comprising an Agrobacterium origin of replication, a first visual marker gene, and a second visual marker gene, wherein the first visual marker gene is a cytokinin autonomy gene, and the second visual marker gene encodes a visually detectable fluorescent protein;
(b) selecting a transformed plant cell and its progeny that do not have visible fluorescence upon illumination with an appropriate light source, wherein fluorescence is associated with read-through and expression of the fluorescent protein gene from the non-T-DNA sequence; and
(c) regenerating a transgenic plant from the selected plant cell.

Optionally, the non-T-DNA sequence employed in the above-described method further comprises an Agrobacterium vir gene, or a polynucleotide encoding one or more Agrobacterium vir proteins, typically 1 to 5 vir proteins, more typically, 1 to 3 vir proteins, most typically 1 to 2 vir proteins.

In yet a further embodiment, the present invention is directed to a method for detecting read-through of non-T-DNA in plants transformed with a T-DNA vector, the method comprising:
(a) transforming a plurality of plant cells with a T-DNA vector comprising:
   (i) a right border, a left border and a T-DNA sequence therebetween, the T-DNA sequence therebetween lacking a functional cytokinin autonomy gene; and
   (ii) a non-T-DNA sequence beyond the left border, the non-T-DNA sequence comprising an Agrobacterium origin of replication, a first visual marker gene, and a second visual marker gene, wherein the first visual marker gene is a cytokinin autonomy gene, and the second visual marker gene encodes a protein that directs the synthesis of a visually detectable chemical compound; and
(b) detecting those transformed plant cells with visible accumulation of the visually detectable chemical compound, which is associated with read-through and expression of the pigment synthesis gene from the non-T-DNA sequence.

Optionally, the non-T-DNA sequence employed in the above-described method further comprises an Agrobacterium vir gene, or a polynucleotide encoding one or more Agrobacterium vir proteins, typically 1 to 5 vir proteins, more typically, 1 to 3 vir proteins, most typically 1 to 2 vir proteins.

In another embodiment, the present invention is directed to a method for producing a transgenic plant containing a polynucleotide of interest but being substantially free of non-T-DNA, the method comprising:
(a) transforming a plurality of plant cells with a T-DNA vector comprising:
   (i) a right border, a left border and a T-DNA sequence therebetween, the T-DNA sequence therebetween containing a polynucleotide of interest and lacking a functional cytokinin autonomy gene; and
   (ii) a non-T-DNA sequence beyond the left border, the non-T-DNA sequence comprising an Agrobacterium origin of replication, a first visual marker gene, and a second visual marker gene, wherein the first visual marker gene is a cytokinin autonomy gene, and the second visual marker gene encodes a protein that directs the synthesis of a visually detectable chemical compound;
(b) selecting a transformed plant cell and its progeny that do not have visible accumulation of the chemical compound, which is associated with read-through and expression of the pigment synthesis gene from the non-T-DNA sequence; and
(c) regenerating a transgenic plant from the selected plant cell.

Optionally, the non-T-DNA sequence employed in the above-described method further comprises an Agrobacterium vir gene, or a polynucleotide encoding one or more Agrobacterium vir proteins, typically 1 to 5 vir proteins, more typically, 1 to 3 vir proteins, most typically 1 to 2 vir proteins.

### BRIEF DESCRIPTION OF SEVERAL VIEWS OF THE DRAWINGS

FIG. 1 discloses a schematic map of the vector pMAXY001 (2002 bp) used in the construction of the binary vectors of the present invention.

FIG. 2 discloses a schematic map of the vector pMAXY002 (2113 bp) used in the construction of the binary vectors of the present invention.

FIG. 3 discloses a schematic map of the vector pMAXY003 (2592 bp) used in the construction of the binary vectors of the present invention.

FIG. 4 discloses a schematic map of the vector pMAXY004 (3214 bp) used in the construction of the binary vectors of the present invention.

FIG. 5 discloses a schematic map of the vector pMAXY005 (4301 bp) used in the construction of the binary vectors of the present invention.

FIG. 6 discloses a schematic map of the vector pMAXY006 (6534 bp) used in the construction of the binary vectors of the present invention.

FIG. 7 discloses a schematic map of the vector pMAXY007 (6682 bp) used in the construction of the binary vectors of the present invention.

FIG. 8 discloses a schematic map of the vector pMAXY008 (7579 bp) used in the construction of the binary vectors of the present invention.

FIG. 9 discloses a schematic map of the vector pMAXY009 (8296 bp) used in the construction of the binary vectors of the present invention.

FIG. 10 discloses a schematic map of the vector pMAXY0010 (8953 bp) used in the construction of the binary vectors of the present invention.

FIG. 11 discloses a schematic map of the vector pMAXY0011 (10457 bp) used in the construction of the binary vectors of the present invention.

FIG. 12 discloses a schematic map of the vector pMAXY0012 (11719 bp) used in the construction of the binary vectors of the present invention.

FIG. 13 discloses a schematic map of the binary vector pMAXY0013 (14521 bp) of the present invention.

FIG. 14 discloses a schematic map of the binary vector pMAXY0014 (14578 bp) of the present invention.

FIG. 15 discloses a schematic map of the vector pMAXY0015 (8795 bp) used in the construction of the binary vectors of the present invention.

FIG. 16 discloses a schematic map of the vector pMAXY0016 (8801 bp) used in the construction of the binary vectors of the present invention.

FIG. 17 discloses a schematic map of the binary vector pMAXY0017 (10305 bp) of the present invention.

FIG. 18 discloses a schematic map of the binary vector pMAXY0018 (11576 bp) of the present invention.

The present invention has multiple aspects. However, the various aspects of the invention are best understood in light of the following definitions.

As used herein, the term “plant” includes reference to whole plants, plant organs (e.g., leaves, stems, roots, etc.), seeds and plant cells and progeny of the same. The class of plants which can be used in the methods of the invention is generally as broad as the class of higher plants amenable to transformation techniques, including both monocotyledonous and dicotyledonous plants.

As used herein, the term “operably linked” includes reference to a functional linkage between a promoter and a second sequence, wherein the promoter sequence initiates and mediates transcription of the DNA sequence corresponding to the RNA sequence which is typically transcribed into a polypeptide. Generally, operably linked means that the nucleic acid sequences being linked are contiguous and, where necessary to join two protein coding regions, contiguous and in the same reading frame.

As used herein, an “expression cassette” is a nucleic acid construct, generated recombinantly or synthetically, with a series of specified nucleic acid elements which permit transcription of a particular nucleic acid in a target cell. The expression cassette can be incorporated into a plasmid, chromosome, mitochondrial DNA, plastid DNA, virus, or nucleic acid fragment. Typically, the expression cassette portion of the expression vector includes, among other sequences, a nucleic acid to be transcribed, and one or more promoters and/or enhancers, typically a promoter with 1 to 4 enhancers, more typically a promoter with 1 to 3 enhancers, most typically a promoter with 1-2 enhancers.

The term “right border,” as used herein, defines a 22 base pair DNA sequence in the case of octopine-type vectors and 25 bp in the case of nopaline-type vectors that is found at the site that initiates T-DNA transfer. A typical right border sequence of the upper strand of an octopine Ti plasmid right border is:

| | |
|---|---|
| 5′-GGCAGGATATATACCGTTGTAA-3′, | (SEQ ID NO:1) |

while a typical right border sequence of the upper strand of a nopaline Ti plasmid right border is:

| | |
|---|---|
| 5′-TGACAGGATATATTGGCGGGTAAAC-3′. | (SEQ ID NO:2) |

See Gielen et al., “The complete nucleotide sequence of the TL-DNA of the Agrobacterium tumefaciens plasmid pTiAch5,” EMBO J. 3:835-846 (1984); and Gielen et al., “The complete nucleotide sequence of the T-DNA region of the plant tumor-inducing Agrobacterium tumefaciens Ti plasmid pTiC58,” J. Exp. Bot. 50:1421-1422 (1999).

Cleavage of the right border in octopine-type vectors occurs between the C and the A at positions 3 and 4 in the bottom strand, and transfer is initiated replicatively in a 5′ to 3′ direction, with replicative displacement of the lower strand. Also included are sequences related to those found in the art as border sequences, and which are sites for virD2-endonucleolytic cleavage.

The term “left border,” as used herein, defines a 22 base pair DNA sequence in the case of octopine-type vectors and 25 bp in the case of nopaline-type vectors that is found at the site that terminates T-DNA transfer. A typical sequence of the upper strand of an octopine Ti plasmid left border is:

| | |
|---|---|
| 5′-GGCAGGATATATTCAATTGTAA-3′, | (SEQ ID NO:3) |

while a typical left border sequence of the upper strand of a nopaline Ti plasmid right border is:

| | |
|---|---|
| 5′-TGGCAGGATATATTGTGGTGTAAAC-3′. | (SEQ ID NO:4) |

See Gielen et al., “The complete nucleotide sequence of the TL-DNA of the Agrobacterium tumefaciens plasmid pTiAch5,” EMBO J. 3:835-846 (1984); and Gielen et al., “The complete nucleotide sequence of the T-DNA region of the plant tumor-inducing Agrobacterium tumefaciens Ti plasmid pTiC58,” J. Exp. Bot. 50:1421-1422 (1999).

Cleavage occurs between the C and the A at positions 3 and 4 in the bottom strand, and transfer initiated at a right border sequence is terminated as a result of the cleavage in the bottom strand (Albright et al., J. Bact. 169:1045-55 (1987)). Also included are sequences related to those found in the art as border sequences, and which are sites for virD2-endonucleolytic cleavage.

As used herein, the term “T-DNA vector” includes reference to any vector that contains at least two T-DNA border elements and which can be used to transfer a nucleic acid sequence positioned between the borders to the genome of a plant cell.

As used herein, the term “T-DNA” includes the DNA fragment that is transferred to the plant cell from a T-DNA vector. The T-DNA is positioned downstream and to the inside of a right border element and upstream and to the inside of a left border element, in either native or recombinant DNA vectors. In a T-DNA vector, the indicated right border and left border elements are generally the only border elements in the vector. However, it is within the scope of the present invention to include multiple copies of the left border to minimize “read-through” of the DNA contained beyond the left border.

As used herein, the term “non-T-DNA” includes any DNA other than the T-DNA in a T-DNA vector of the invention. In a T-DNA vector, the non-T-DNA is positioned upstream and to the outside of a right border and downstream and to the outside of a left border.

In its first aspect, the present invention is directed to an isolated T-DNA vector for identifying read-through of non-T-DNA, the T-DNA vector comprising i) a right border, a left border and a T-DNA sequence positioned therebetween, the T-DNA sequence therebetween lacking a functional cytokinin autonomy gene (such as found in the wild-type Ti or Ri plasmids); and ii) a non-T-DNA sequence beyond the left border, the non-T-DNA sequence comprising an Agrobacterium origin of replication, and a first visual marker gene, the first visual marker gene being a cytokinin autonomy gene.

Optionally, the non-T-DNA sequence further comprises an Agrobacterium vir gene, or a polynucleotide encoding one or more Agrobacterium vir proteins, typically 1 to 5 vir proteins, more typically, 1 to 3 vir proteins, most typically 1 to 2 vir proteins.

In the vector of the present invention, the DNA sequence positioned between the right border and the left border typically comprises either: i) a nucleic acid sequence or a gene, encoding a protein of interest (collectively “a gene of interest”); ii) an expression cassette for receiving the nucleic acid sequence or the gene of interest; or iii) the expression cassette containing the nucleic acid sequence or the gene of interest. The expression cassette comprises a transcriptional initiation region linked to the nucleic acid or gene of interest. Such an expression cassette is provided with a plurality of restriction sites for insertion of one or more nucleic acid sequences of genes of interest to be under the transcriptional regulation of the regulatory regions.

Genes of interest are reflective of the commercial markets and interests of those involved in the development of the crop. Examples of some genes of interest include genes encoding important traits for agronomics, insect resistance, disease resistance, herbicide resistance, sterility, color, taste, shape, and grain characteristics.

In one embodiment, the genes of interest encode proteins that affect agronomically important traits such as oil, starch and protein content. Modifications include increasing content of oleic acid, saturated and unsaturated oils, increasing levels of lysine and sulfur and providing essential amino acids, and also modification of starch. Hordothionin protein modifications are described in European Patents WO96/38563, WO94/16078 and WO96/38562, the disclosures of which are incorporated herein in their entirety by reference. Another example is a lysine and/or sulfur rich seed protein encoded by the soybean 2S albumin described in U.S. Ser. No. 08/618,911 filed Mar. 20, 1996, and the chymotrypsin inhibitor from barley (Williamson et al., Eur. J. Biochem. 165:99-106 (1987), the disclosures of each are incorporated by reference. Derivatives of the following genes can be made by site-directed mutagenesis to increase the level of preselected amino acids in the encoded polypeptide. For example, the gene encoding the barley high lysine polypeptide (BHL) is derived from barley chymotrypsin inhibitor, U.S. Ser. No. 08/740,682 filed Nov. 1, 1996, and PCT/US97/20441 filed Oct. 31, 1997, the disclosures of each are incorporated herein by reference. Other proteins include methionine-rich plant proteins such as from sunflower seed (Lilley et al., Proceedings of the World Congress on Vegetable Protein Utilization in Human Foods and Animal Feedstuffs, Applewhite, H. (ed.); American Oil Chemists Soc., Champaign, Ill., 497-502 (1989); corn (Pedersen et al., J. Biol. Chem. 261:6279 (1986); Kirihara et al., Gene 71:359 (1988); and rice (Musumura et al., Plant Mol. Biol. 12:123 (1989). These examples are herein incorporated by reference. Other agronomically important genes encode latex, Floury 2, growth factors, seed storage factors and transcription factors.

Other genes of interest may encode proteins that confer resistance to pests, such as the corn rootworm or the European corn borer, which decrease crop yields. An example of an insect resistance gene is the gene encoding the crystal or toxic protein of Bacillus thuringiensis. This gene has been isolated, characterized and successfully used to lessen ECB infestation (U.S. Pat. No. 5,366,892, Foncerrada et al., “Gene Encoding a Coleopteran-active Toxin”). Other examples of genes useful in insect resistance include those encoding secondary metabolites and natural plant toxins.

Yet other genes of interest are disease resistance genes, and include detoxification genes, such as genes that encode proteins that detoxify fumonosin or other toxins. Fumonisin-detoxifying resistance genes are used to transform plant cells normally susceptible to Fusarium or other toxin-producing fungi as described in U.S. Pat. No. 5,792,931. Other examples are genes conferring viral resistance and genes encoding antimicrobial peptides.

Further genes of interest are herbicide resistance genes. These genes are exemplified by genes coding for resistance to sulfonylurea-type herbicides, which act to inhibit the action of acetolactate synthase (ALS); genes coding for resistance to the herbicides phosphinothricin or BASTA®, which act to inhibit action of glutamine synthase; genes encoding for resistance to the herbicide glyphosate; genes coding for resistance to the herbicide cyanamide; and by other such genes well known in the art. Suitable herbicide resistance genes include the bar gene of Streptomyces hygroscopicus, which encodes phosphinothricin acetyl transferase and confers resistance to the herbicide (BASTA®) (WO 97/05829; U.S. Pat. No. 5,489,520); the neomycin phosphotransferase II, or nptII gene, which encodes resistance to the antibiotics kanamycin and genitimicin (Fraley et al., PNAS U.S.A. 80:4803 (1983)); the modified ALS gene (containing mutations such as the S4 and/or Hra mutations) which encodes resistance to the herbicide chlorsulfuron (U.S. Pat. No. 5,013,659); the glyphosate N-acetyl transferase gene, such as GAT r9 22-18c5, which encodes resistance to the herbicide glyphosate (ROUND-UP®); the altered 5-enolpyruvyl-3-phosphoshikimate (EPSP) synthase gene, which encodes resistance to the herbicide glyphosate (ROUND-UP®) (U.S. Pat. No. 5,554,798); the nucleic acid sequence encoding for cyanamide hydratase (CAH), which encodes resistance to the herbicide cyanamide (U.S. Pat. No. 6,660,910 and Maier-Greiner et al., PNAS USA 88: 4260-4264, 1991).

Other genes of interest that affect the quality of an agricultural plant are reflected in traits such as levels and types of oils, saturated and unsaturated, quality and quantity of essential amino acids, and levels of cellulose. In corn, modified hordothionin proteins are described in WO96/38563, WO94/16078 and WO96/38562; and U.S. Pat. No. 5,703,409 issued Dec. 30, 1997. These publications provide descriptions of modifications of proteins for desired purposes. Commercial traits can also be encoded on a gene or genes which could increase, for example, starch for ethanol production, or provide expression of proteins. Another important commercial use of transformed plants is the production of polymers and bioplastics, such as described in U.S. Pat. No. 5,602,321, issued Feb. 11, 1997. Genes such as β-ketothiolase, PHBase (polyhydroxyburyrate synthase) and acetoacetyl-CoA reductase (see Schubert et al., J. Bacteriol. 170(12):5837-5847 (1988)) facilitate expression of polyhyroxyalkanoates (PHAs).

It should be pointed out that all of the above-cited references and the references to follow are expressly incorporated herein by reference in their entirety.

It is also within the scope of the present invention that the “gene of interest” within the expression cassette be an antisense gene, a gene fragment, or a nucleic acid designed to trigger RNAi-type silencing.

In the vector of the present invention, a component of the non-T-DNA sequence is an Agrobacterium origin of replication. A suitable origin of replication is the ori V trfA region of the broad host range plasmid RK2. Another suitable origin of replication is the pVS1 ori, which is obtained from the pVS1 plasmid of Pseudomonas (Itoh and Haas, Gene 36:27-36 (1985)). Other origins of replication that function well for plasmid maintenance in Agrobacterium have been described in Hellens et al., Trends in Plant Science 5:446-451 (2000).

Another component of the non-T-DNA sequence is the first visual marker gene, which is a cytokinin autonomy gene. In the wild-type Ti plasmid and in some T-DNA vectors, the cytokinin autonomy gene is a component of the T-DNA sequence. Applicants have found that it is advantageous to remove the cytokinin autonomy gene from its usual location in the T-DNA sequence, and to instead employ a cytokinin autonomy gene in the non-T-DNA portion of the vector. The cytokinin autonomy gene is a visual marker to indicate transfer beyond the left border. Any read-through of the cytokinin autonomy gene present in the non-T-DNA region causes visible phenotypic changes such as abnormal cell proliferation, accelerated or deeper “greening,” delayed senescence, or “shooty” tumor formation (Taiz and Zeiger, Plant Physiology, E. Brady (ed.) The Benjamin/Cummings Publishing Company, Inc., 1991). Preferably, the cytokinin autonomy gene is obtained from A. tumefaciens. Suitable sources for the cytokinin autonomy gene from A. tumefaciens are disclosed in U.S. Pat. No. 6,051,757; U.S. Pat. No. 6,359,197 (Amasino); U.S. Pat. No. 6,294,715 (Matsunaga); Akiyoshi, et al., PNAS 81:5994-5998 (1984); or Barry et al., PNAS 81:4776-4780 (1984), all of which are incorporated herein by reference. DNA sequences of cytokinin autonomy genes, which are also referred to as “IPT” sequences or as “tmr” or “tzs” genes, are disclosed in Crespi et al., EMBO J. 11:795-804 (1992); Kamp et al., Nucleic Acids Res. 11:6211-6223 (1983); Strabala et al., Mol. Gen. Genet. 216:388-394 (1989); Powell et al., Nucl. Acids Res. 14:2555-2565 (1986); Akiyoshi et al., Nucl. Acids Res. 17:8886 (1989); and Lichter et al., J. Bacteriol. 177:4457-4465 (1995), all of which are incorporated herein by reference.

In addition, certain plant genes may also function as “cytokinin autonomy genes” when over-expressed in plant cells. See Kakimoto, Science 274:982-985 (1996); Banno et al., Plant Cell 13:2609-2618 (2001); and Hewelt et al., Planta 210:884-889 (2000).

In another embodiment, the non-T-DNA sequence of the T-DNA vector of the present invention comprises a vir gene. The vir region of the Agrobacterium Ti plasmid is composed of seven major loci (virA, virB, virC, virD, virE, virG, and virH. The protein products of these genes respond to specific compounds secreted by the wounded plant and generate the T-DNA strand that is to be transferred into the host cell. See Sheng and Citovsky, Plant Cell 8:1699-1710 (1996). For example, the VirD1 and VirD2 proteins recognize and nick the right and left border regions at specific locations and mediate the transfer of the T-DNA into the infected plant cell for integration into the genome. Where the vector of the present invention has a plurality of left border sequences, there are a plurality of opportunities for the VirD proteins to nick a left border and minimize “beyond the border” DNA transfer. Providing additional copies of a vir gene or vir region in the non-T-DNA portion of the present invention may enhance the formation of T-strands and improve the transformation efficiency of plant cells. See Tang, Plant Cell Rep. 21:555-562 (2003); and Liu et al., Plant Mol. Biol. 20:1071-1087 (1992).

Generally, the vectors described herein were made using laboratory procedures in recombinant DNA technology that are well-known and commonly employed in the art. Standard techniques were used for cloning, DNA and RNA isolation, amplification and purification. Generally, the enzymatic reactions involving DNA ligase, DNA polymerase, restriction endonucleases and the like were performed according to the manufacturer's specifications. These techniques and various other techniques were generally performed according to Sambrook et al., Molecular Cloning—A Laboratory Manual, Cold Spring Harbor Laboratory, Cold Spring Harbor, N.Y., (1989), the whole of which is incorporated herein by reference.

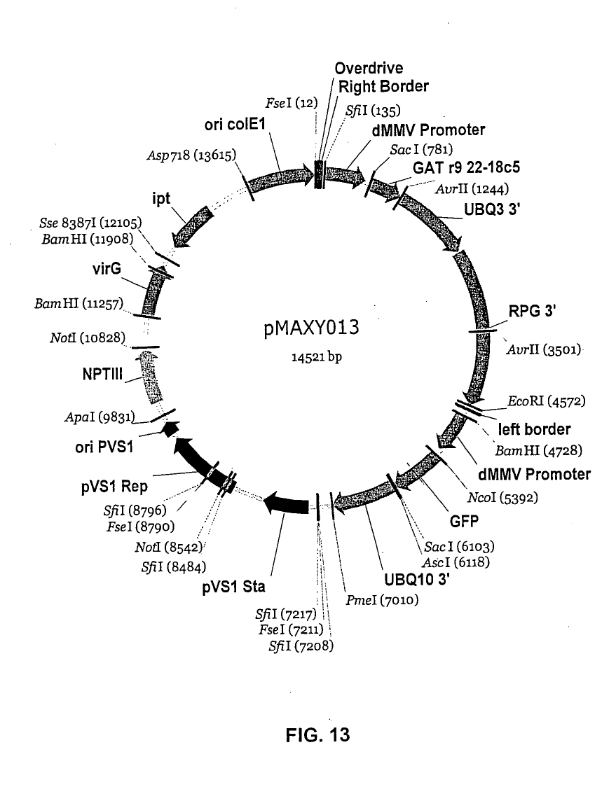

A detailed description of how to construct three of the isolated T-DNA vectors of the present invention is disclosed in Examples 1 through 7 provided herein. These vectors are pMAXY013 (14521 bp), pMAXY017 (10305 bp), and pMAXY018 (11576 bp), the schematic maps of which are disclosed in FIGS. 13, 17 and 18, respectively. The vector pMAXY018 is identical to pMAXY017, except for the addition of an Agrobacterium vir gene (virG) into the binary vector backbone.

In another embodiment, the non-T-DNA sequence of the above-described vector also comprises a second visual marker gene, such as a gene encoding a visually detectable fluorescent protein. Any transformant having “read-through” that causes expression of the second visual marker gene will be visually detectable by illuminating the transformant with an excitation frequency for the fluorescent protein and checking the illuminated transformants for the appropriate color of fluorescence. The excitation and fluorescent wavelengths for various fluorescent proteins are disclosed in Table 1 herein. Thus, the vector of the present invention allows for the early detection and elimination of those transformants wherein read-through has occurred.

Alternatively, the second visual marker gene encodes a protein whose expression triggers the production of a visually detectable chemical compound. Examples of such genes include those involved in the regulation or biosynthesis of anthocyanin, carotenoid, or indigo pigments. See Walbot et al., Basic Life Sci. 41:183-188 (1987); Lloyd et al., Science 258:1773-1775 (1992); Borevitz et al., Plant Cell 12:2383-2394 (2000); Holton, Drug Metabol Drug Interact. 12:359-368 (1995); Sandmann, Arch Biochem Biophys. 385:4-12 (2001); Mann et al., Nat. Biotechnol. 18:888-892 (2000); Minami et al., Plant Cell Physiol. 41:218-225 (2000); and Shewmaker et al., The Plant Journal 20: 401-412 (1999). Any transformant having “read-through” that causes expression of these marker genes will be visually detectable by examining the transformant under visible light and checking for the expected color of the illuminated chemical compound(s). Thus, the vectors of the present invention provide various embodiments that allow for the early detection and elimination of those transformants wherein read-through has occurred.

Any fluorescent protein can be used in the present invention, including proteins that fluoresce due to intramolecular rearrangements or the addition of cofactors that promote fluorescence. Suitable fluorescent proteins are known in the art and include green fluorescent protein, blue fluorescent protein, yellow-green fluorescent protein, yellow fluorescent protein, red fluorescent protein, cyan fluorescent protein and mutants thereof. A green fluorescent protein (“GFP”) is a protein that emits green light, a blue fluorescent protein (“BFP”) is a protein that emits blue light, a yellow fluorescent protein (“YFP”) is one that emits yellow light, a red fluorescent protein is a protein (“RFP”) that emits red light, and a cyan fluorescent protein (“CFP”) is one that emits a greenish-blue light.

A preferred detectable fluorescent protein is green fluorescent protein. As used herein, “green fluorescent protein” (GFP) refers to one of the class of proteins typically from marine organisms that emit green light when activated. Suitable green fluorescent proteins have been isolated and characterized from marine organisms such as the Pacific Northwest jellyfish, Aequorea victoria, the sea pansy, Renilla reniformis, and Phialidium gregarium. A preferred green fluorescent protein, such as the GFP of Aequorea, can be employed in a variety of cells and requires no substrate to fluoresce. A transformed cell containing the green fluorescent protein fluoresces under various conditions, including when excited with blue light of about 450 nm to about 490 nm. See, e.g., Levine et al., Comp. Biochem. Physiol. 72B:77-85 (1982) and U.S. Pat. No. 6,667,153; which are incorporated herein by reference for its disclosure of the structure and manipulation of the DNA sequence encoding Phialidium gregarium green fluorescent protein.

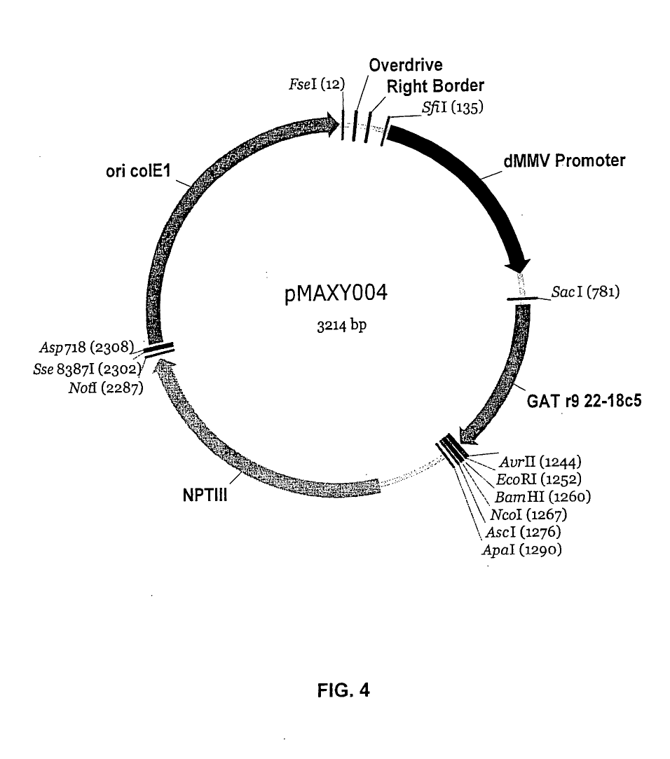

In a preferred embodiment, the green fluorescent protein is the 238-residue GFP of the jellyfish Aequorea victoria or a variant of this GFP. The DNA sequence encoding the wild-type GFP of the jellyfish Aequorea Victoria is disclosed in FIGS. 4a and 4b of U.S. Pat. No. 5,958,713 (Thastrup et al.); Prasher, “Primary structure of the Aequorea victoria green-fluorescent protein,” Gene 111: 229-233 (1992) (Gen Bank Accession No. M62653); and Inouye et al., FEBS Lett 351(2-3): 277-280 (1994) with (GenBank accession number L29345), the sequences of which are incorporated herein by reference.

Those of skill in the art know the coding sequences for a variety of useful variants of Aequorea GFP. See, e.g., U.S. Pat. Nos. 6,667,153; 5,625,048; 5,804,287; and 5,998,204; PCT publication WO 96/23810; Cormack et al., “FACS-optimized mutants of the green fluorescent protein (GFP)”, Gene: 173, 33-38 (1996) disclosing the coding of GFP mutant 3 (GenBank accession number U73901); Crameri et al., “Improved Green Fluorescent Protein by Molecular Evolution Using DNA Shuffling,” Nature Biotechnology 14: 315-319 (1996); Heim et al., “Improved Green Fluorescence,” Nature 373: 663-664 (1995); and Heim et al., “Engineering green fluorescent protein for improved brightness, longer wavelengths and fluorescence resonance energy transfer,” Current Biology 6:178-182 (1996), all of which are incorporated herein by reference for their disclosure of the structure and manipulation of green fluorescent proteins and polynucleotides that encode them. Such variants can be employed in the vectors and methods of the present invention. Methods for manipulating the coding sequence of the Aequorea GFP, and plasmids that include the GFP coding sequence, are known in the art and are described in, for example, U.S. Pat. No. 6,667,153, and in Matthysse et al., FEMS Microbiology Letters 145:87-94 (1996), both of which are incorporated herein by reference.

Useful variants of wild-type Aequorea GFP have amino acid substitutions at one or more of residue positions: 26, 64, 65, 66, 68, 72, 73, 99, 100, 123, 127, 145, 146, 147, 148, 149, 153, 154, 163, 164, 167, 185, 202, 203, 208, 212, 235, and 238. Known amino acid substitutions that produce useful variants of Aequorea GFP include one or more of the following: K26R, F64L, F64C, F64M, S65A, S65C, S65G, S65L, S65I, S65T, S65V, Y66H, Y66F, Y66W, V68L, V68C, S72A, S73P, F99S, F100S, I123V, K127E, Y145F, Y145H, N1461, N147Y, H148R, N149K, M153T, M154T, V163A, N164H, I167T, I167V, Q185R, S202F, T203F, T203H, T203I, T203L, T203W, T203Y, S208L, N212K, E235D, K238E, and K238N. Useful variants include those in which the residue(s) at one or more of positions 64, 68, or 72 is varied either individually or together with the residue at position 65. The variants of wild-type Aequorea GFP listed in this paragraph, and polynucleotides encoding them, can be employed in the vectors and methods of the present invention.

Additional useful changes in the excitation and emission spectra of GFP result from combinations of amino acid substitutions such as S73P, F100S, K127E, N147Y, M154T, V164A, and E235D; F100S, M154T, V164A, Q185R, and E235D; and F100S, M154T, and V164A.

Variants, such as Y66H and Y66W (Tsien, Ann. Rev. Biochem. 67:509 (1998), Heim et al., Proc. Natl. Acad. Sci. (USA) 91:12501 (1994) result in blue-shifted absorbance and emission maxima. See also Wachter et al., Biochemistry 36:9759-9765 (1997). The variant green fluorescent proteins listed herein, and polynucleotides encoding them, can be employed in the vectors and methods of the invention.

Some Aequorea-related engineered versions of GFP are described in Table 1.

TABLE 1

| | | | | Extinction |
|---|---|---|---|---|
| | | | | Coefficient |
| | | Excitation Emission | | (M. sup. −1 |
| Clone | Mutation(s) | max (nm) | max (nm) | cm. sup. −1) |
| Wild-type | none | 395 (475) | 508 | 21,000 (7,150) |
| P4 | Y66H | 383 | 447 | 13,500 |
| P4-3 | Y66H; Y145F | 381 | 445 | 14,000 |
| P4-3E | Y66H; Y145F | 384 | 448 | 22,000 |
| | V163A | | | |
| W7 | Y66W; N146I; | 433 (453) | 475 (501) | 18,000 (17,100) |
| | M153T; V163A; | | | |
| | N212K | | | |
| W2 | Y66W; I123V; | 432 (453) | 430 | 10,000 (9,600) |
| | Y145H; H148R; | | | |
| | M153T; V163A; | | | |
| | N212K | | | |
| S65T | S65T | 489 | 511 | 39,200 |
| P4-1 | S65T; M153A; | 504 (396) | 514 | 14,500 (8,600) |
| | K238E | | | |
| S65A | S65A | 471 | 504 | |
| S65C | S65C | 479 | 507 | |
| S65L | S65I | 484 | 510 | |
| Y66F | Y66F | 360 | 442 | |
| Y66W | Y66W | 458 | 480 | |
| 10C | S65G | 514 | 527 | 83,400 |
| W1B | F64L; S65T; | 434 (452) | 476 (505) | 32,500 |
| | Y66W; N146I; | | | |
| | M153T; V163A; | | | |
| | N212K | | | |
| Emerald | S65T; S72A; | 487 | 509 | 57,500 |
| | N149K; M153T; | | | |
| | I167T | | | |
| Sapphire | S72A; Y145F; | 395 | 511 | 29,000 |
| | T203I | | | |
| Topaz | S65G; S72A; | 514 | 527 | 94,500 |
| | K79R; T203Y | | | |
| EGFP | F64L; S65T | 588 | 507 | 55,900 |

Numerous coding sequences for wild-type and useful variants of Aequorea green fluorescent protein are commercially available, for example, from Clontech (Palo Alto, Calif.) and Quantum Biotechnologies (Montreal, Quebec, Canada). These suppliers provide the coding sequence in cloning and expression vectors. The coding sequences encode variants of green fluorescent protein that fluoresce blue, cyan, green, and yellow-green, and that can provide brighter fluorescence compared to the wild-type protein. Such variants can be employed in the compositions and methods of the present invention.

Fluorescent proteins having other colors are also known in the art. The DNA sequence encoding several yellow fluorescent proteins is disclosed in Baldwin et al., Biochemistry 29: 5509-5515 (1990), the whole of which is hereby incorporated by reference. Other sources of yellow fluorescent proteins include phycobiliproteins from marine cyanobacteria such as Synechococcus, e.g., phycoerythrin and phycocyanin; oat phytochromes from oat reconstructed with phycoerythrobilin; or the Propionibacterium freudenreich uroporphyrinogen III methyltransferase (cobA) gene product.

The blue fluorescent protein is one of the classes of proteins, typically from marine organisms, that emit blue-green light when activated. See Karatani et al., Photochem. Photobiol. 55: 293-299 (1992).

A suitable red fluorescent protein, found in the IndoPacific sea anemone relative Discosoma species, is commercially available from Clontech (Palo Alto, Calif.) in cloning and expression vectors. See Wilbanks et al., J. Biol. Chem. 268:1226-1235 (1993); which are incorporated herein by reference for their disclosure of the structure and manipulation of fluorescent proteins and polynucleotides that encode them. See also Li et al., Biochemistry 34:7923-7930 (1995) and Wildt et al. Nat. Biotech. 17:1175-1178 (1999).

In addition, “GFP-like” proteins and/or nonfluorescent chromoproteins from Anthozoans could potentially be adapted for use with the present invention. See Verkhusha and Lukyanov, Nat. Biotechnol. 22:289-296 (2004).

The promoters employed in the instant invention as part of the expression cassettes of the invention (e.g., those comprising the polynucleotides of interest, and the like) preferably function effectively in the target cells for Agrobacterium-mediated DNA delivery. The chosen promoters may function effectively in cells other than the target cells. One useful class of promoters is that generally characterized as constitutive. Examples of constitutive promoters include the FLt promoter from mirabilias mosaic virus (MMV) (Dey and Matai, Plant Mol. Biol. 40:771-782, 1999); the FLt promoter from peanut chlorotic streak virus (PCSV) (Maiti and Shepherd, Biochem. Biophys. Res. Comm. 244:440-444, 1998); the Fit promoter from strawberry vein banding virus (SVBV) (Wang et al., Virus Genes 20:11-17, 2000); the 34S promoter from figwort mosaic virus (FMV) (Sanger et al., Plant Mol. Biol. 14:433-443, 1990; Maiti et al., Transgenic Res. 6:143-156, 1997); the 35S promoter from cauliflower mosaic virus (CaMV) (Odell et al., Nature 313:810-812, 1985); and the promoters from such genes as rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)). Another useful constitutive promoter is the ALS promoter, XbaI/NcoI fragment 5′ to the Brassica napus ALS3 structural gene (or a nucleotide sequence similarity to said Xba1/NcoI fragment). See PCT application WO96/30530.

Alternatively, the plant promoter may direct expression of the operably linked nucleic acid in a specific tissue or may be otherwise under more precise environmental or developmental control. Examples of environmental conditions that may effect transcription by inducible promoters include anaerobic conditions, elevated temperature, or the presence of light. Such promoters are referred to here as “inducible” or “tissue-specific” promoters. One skilled in the art recognizes that a tissue-specific promoter may drive expression of operably linked sequences in tissues other than the target tissue. Thus, as used herein, a tissue-specific promoter is one that drives expression preferentially in the target tissue, but may also lead to some expression in other tissues as well.

Examples of promoters under developmental control include promoters that initiate transcription only (or primarily only) in certain tissues, such as fruit, seeds, or flowers.

Other useful promoters are those that are effective in callus tissues, as such are often derived during plant transformation procedures. Other types of promoters can be selected for their activity in embryogenic tissue, where such tissues are being used as the explant for transformation. The “double” or enhanced mirabilias mosaic virus (dMMV) promoter (Dey and Maiti, Transgenics 3:61-70, 1999) is a preferred promoter for use with the present invention as it is active in all tissue types, so that a single vector can be used with any plant explant and transformation type.

The present invention is practiced with a range of transformation methods, including any of the methods within the broad classes of organogenic and embryogenic regeneration methods. Organogenic methods are those in which shoots are derived either directly from a standard plant tissue explant, or from callus tissue that arises from a standard plant tissue explant during the transformation process, and then whole plants are derived from those shoots. Embryogenic methods are those in which specific tissue types capable of producing embryos are transformed, and then embryos are regenerated.

The T-DNA vectors of the present invention are DNA constructs which can be cloned and/or synthesized by any number of standard techniques. Expression cassette within the vectors (e.g., those encoding a desired polypeptide, the lethal polypeptide, and the like) will typically comprise transcriptional and translational initiation regulatory sequences, which will direct the transcription of the polynucleotide encoding the polypeptide in the intended tissues of the transformed plant. The T-DNA vectors are introduced into the genome of the desired plant host using a conventional Agrobacterium host (e.g., A. tumefaciens, A. rhizogenes). The virulence functions of the Agrobacterium host will direct the insertion of the construct and adjacent marker into the plant cell DNA when the cell is infected by the bacteria. Agrobacterium-mediated transformation techniques, including the use of binary vectors, are well-described in the scientific literature. See, e.g., Horsch et al., Science 233:496-498 (1984), and Fraley et al., Proc. Natl. Acad. Sci. USA 80:4803 (1983).

In another embodiment, the present invention is directed to a method for detecting read-through of non-T-DNA in plants transformed with a T-DNA vector, comprising:
(a) transforming a plurality of plant cells with a T-DNA vector comprising:
   (i) a right border, a left border and a T-DNA sequence therebetween, the T-DNA sequence therebetween lacking a functional cytokinin autonomy gene; and
   (ii) a non-T-DNA sequence beyond the left border, the non-T-DNA sequence comprising an Agrobacterium origin of replication and a first visual marker gene, the first visual marker gene being a cytokinin autonomy gene; and
(b) detecting those plant cells showing a “shooty” or other characteristic phenotype which is associated with read-through and expression of the cytokinin autonomy gene from the non-T-DNA sequence. Optionally, the non-T-DNA sequence employed in the above-described method further comprises an Agrobacterium vir gene, or a polynucleotide encoding one or more Agrobacterium vir proteins, typically 1 to 5 vir proteins, more typically, 1 to 3 vir proteins, most typically 1 to 2 vir proteins. In another embodiment of the above method, the non-T DNA sequence further comprises a gene encoding a second visual marker, such as a gene encoding a fluorescent protein, or a gene encoding a protein involved in the regulation or biosynthesis of anthocyanin, carotenoid, or indigo pigments. Preferably, the second visual marker is a gene encoding a fluorescent protein, more preferably, a green fluorescent protein.

In another aspect, the present invention is directed to a method for producing a transgenic plant containing a polynucleotide of interest, the method comprising:
(a) transforming a plurality of plant cells with a T-DNA vector comprising:
   (i) a right border, a left border and a T-DNA sequence therebetween, the T-DNA sequence therebetween lacking a functional cytokinin autonomy gene; and
   (ii) a non-T-DNA sequence beyond the left border, the non-T-DNA sequence comprising an Agrobacterium origin of replication and a first visual marker gene, the first visual marker gene being a cytokinin autonomy gene;
(b) selecting a transformed plant cell and its progeny which integrate and express the T-DNA sequence and do not visually manifest abnormal cell proliferation, accelerated or deeper “greening,” delayed senescence, the formation of “shooty” tumors, or other phenotypes associated with read-through and expression of cytokinin autonomy gene from the non-T-DNA sequence; and
(c) regenerating a transgenic plant from the selected plant cell.

In a further embodiment, the present invention is directed to a method for detecting read-through of non-T-DNA in plants transformed with a T-DNA vector, the method comprising:
(a) transforming a plurality of plant cells with a T-DNA vector comprising:
   (i) a right border, a left border and a T-DNA sequence therebetween, the T-DNA sequence therebetween lacking a functional cytokinin autonomy gene; and
   (ii) a non-T-DNA sequence beyond the left border, the non-T-DNA sequence comprising an Agrobacterium origin of replication, a first visual marker gene, and a second visual marker gene, wherein the first visual marker gene is a cytokinin autonomy gene and the second visual marker gene encodes a visually detectable fluorescent protein; and
(b) detecting those transformed plant cells with visible fluorescence upon illumination with an appropriate light source, wherein fluorescence is associated with read-through and expression of the fluorescent protein gene from the non-T-DNA sequence.

Optionally, the non-T-DNA sequence employed in the above-described method further comprises an Agrobacterium vir gene, or a polynucleotide encoding one or more Agrobacterium vir proteins, typically 1 to 5 vir proteins, more typically, 1 to 3 vir proteins, most typically 1 to 2 vir proteins.

In another embodiment, the present invention is directed to a method for producing a transgenic plant containing a polynucleotide of interest but being substantially free of non-T-DNA, the method comprising:
(a) transforming a plurality of plant cells with a T-DNA vector comprising:
   (i) a right border, a left border and a T-DNA sequence therebetween, the T-DNA sequence therebetween lacking a functional cytokinin autonomy gene; and
   (ii) a non-T-DNA sequence beyond the left border, the non-T-DNA sequence comprising an Agrobacterium origin of replication, a first visual marker gene, and a second visual marker gene, wherein the first visual marker gene is a cytokinin autonomy gene, and the second visual marker gene encodes a visually detectable fluorescent protein;
(b) selecting a transformed plant cell and its progeny that do not have visible fluorescence upon illumination with an appropriate light source, wherein fluorescence is associated with read-through and expression of the fluorescent protein gene from the non-T-DNA sequence; and
(c) regenerating a transgenic plant from the selected plant cell.

Optionally, the non-T-DNA sequence employed in the above-described method further comprises an Agrobacterium vir gene, or a polynucleotide encoding one or more Agrobacterium vir proteins, typically 1 to 5 vir proteins, more typically, 1 to 3 vir proteins, most typically 1 to 2 vir proteins.

In yet a further embodiment, the present invention is directed to a method for detecting read-through of non-T-DNA in plants transformed with a T-DNA vector, the method comprising:
(a) transforming a plurality of plant cells with a T-DNA vector comprising:
   (i) a right border, a left border and a T-DNA sequence therebetween, the T-DNA sequence therebetween lacking a functional cytokinin autonomy gene; and
   (ii) a non-T-DNA sequence beyond the left border, the non-T-DNA sequence comprising an Agrobacterium origin of replication, a first visual marker gene, and a second visual marker gene, wherein the first visual marker gene is a cytokinin autonomy gene, and the second visual marker gene encodes a protein that directs the synthesis of a visually detectable chemical compound; and
(b) detecting those transformed plant cells with visible accumulation of the visually detectable chemical compound, which is associated with read-through and expression of the pigment synthesis gene from the non-T-DNA sequence.

Optionally, the non-T-DNA sequence employed in the above-described method further comprises an Agrobacterium vir gene, or a polynucleotide encoding one or more Agrobacterium vir proteins, typically 1 to 5 vir proteins, more typically, 1 to 3 vir proteins, most typically 1 to 2 vir proteins.

In another embodiment, the present invention is directed to a method for producing a transgenic plant containing a polynucleotide of interest but being substantially free of non-T-DNA, the method comprising:
(a) transforming a plurality of plant cells with a T-DNA vector comprising:
   (i) a right border, a left border and a T-DNA sequence therebetween, the T-DNA sequence therebetween containing a polynucleotide of interest and lacking a functional cytokinin autonomy gene; and
   (ii) a non-T-DNA sequence beyond the left border, the non-T-DNA sequence comprising an Agrobacterium origin of replication, a first visual marker gene, and a second visual marker gene, wherein the first visual marker gene is a cytokinin autonomy gene, and the second visual marker gene encodes a protein that directs the synthesis of a visually detectable chemical compound;
(b) selecting a transformed plant cell and its progeny that do not have visible accumulation of the chemical compound, which is associated with read-through and expression of the pigment synthesis gene from the non-T-DNA sequence; and
(c) regenerating a transgenic plant from the selected plant cell.

Optionally, the non-T-DNA sequence employed in the above-described method further comprises an Agrobacterium vir gene, or a polynucleotide encoding one or more Agrobacterium vir proteins, typically 1 to 5 vir proteins, more typically, 1 to 3 vir proteins, most typically 1 to 2 vir proteins. Agrobacterium-mediated genetic transformation of plants involves several steps. The first step, in which the Agrobacterium and plant cells are first brought into contact with each other, is generally called “inoculation.” Following the inoculation step, the Agrobacterium and plant cells/tissues are usually grown together for a period of several hours to several days or more under conditions suitable for growth and T-DNA transfer. This step is termed “co-culture” or “co-cultivation.” Following co-culture and T-DNA delivery, the plant cells are often treated with bacteriocidal and/or bacteriostatic agents to kill the Agrobacterium. If this is done in the absence of any selective agents to promote preferential growth of transgenic versus non-transgenic plant cells, then this is typically referred to as the “delay” step. If done in the presence of selective pressure favoring transgenic plant cells, then it is referred to as a “selection” step. When a “delay” is used, it is followed by one or more “selection” steps. Both the “delay” and “selection” steps typically include bacteriocidal and/or bacteriostatic agents to kill any remaining Agrobacterium cells because the growth of Agrobacterium cells is undesirable after the infection (inoculation and co-culture) process.

The methods of the present invention are useful with any plant to which Agrobacterium species can transfer DNA. Thus, the methods of the present invention are useful for the transformation of dicotyledonous plants, which are the natural target for Agrobacterium infection. More recently, the species of plants subject to transformation with Agrobacterium has come to include monocotyledonous plants, such as rice and maize. See Hiei et al., Plant J. 6:271-282 (1994), and U.S. Pat. No. 5,591,616, issued Jan. 7, 1997, all of which are incorporated herein by reference.

Suitable plant genera that can be used with the present invention include, for instance, Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Gossypium, Pisum, Phaseolus, Lolium, Oryza, Zea, Avena, Hordeum, Secale, Triticum, Sorghum and Datura.

The present invention can be practiced with any explant for which transformation is possible. This includes, but is not limited to, leaf discs, stem explants, floral tissues, root tissue, embryogenic tissues, callus tissues, protoplasts, or suspension cells.

A first vector of the present invention is pMAXY013 (14521 bp), as shown in FIG. 13, having a T-DNA sequence flanked by a right border (with an overdrive element) on the 5′ end and a left border on the 3′ end. The T-DNA sequence is comprised of, in the 5′ to 3′ direction, a constitutive plant promoter (the duplicated mirabilis mosaic virus or dMMV promoter), a gene of interest which has commercial value and serves as a selectable marker in tissue culture (the glyphosate-N-acetyltransferase, or GAT variant r9 22-18c5, gene) and two 3′ termination regions (the polyubiquitin 3 gene terminator, or UBQ3 3′, and a ribosomal protein gene terminator, or RPG 3′). Downstream of the left border in the non-T-DNA portion of the vector is a visual marker expression cassette comprised of the dMMV promoter, a polynucleotide encoding a green fluorescent protein (GFP), and a plant 3′ termination region (the polyubiquitin 10 gene terminator, or UBQ10 3′). Following this is a region that allows plasmid replication and maintenance in Agrobacterium (the Pseudomonas PVS1 sta gene, Rep gene, and origin of replication), a kanamycin resistance gene (NPTIII) for bacterial selection, a gene encoding one of the Agrobacterium virulence proteins (virG), another visual marker gene comprised of the Agrobacterium isopentenyl transferase (ipt), or cytokinin autonomy, gene in reverse orientation, and an E. coli origin of replication (ori ColE1).

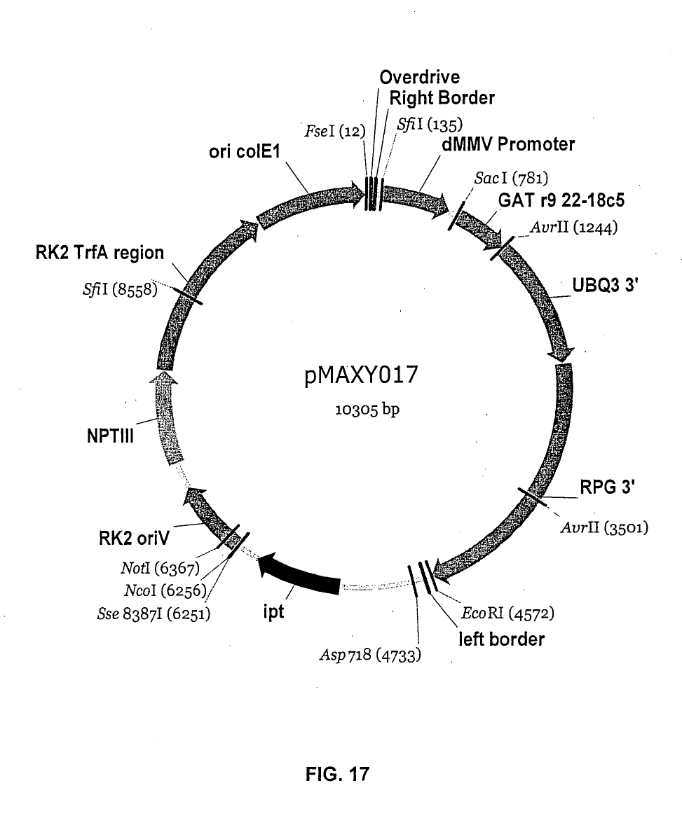

A second vector of the present invention is pMAXY017 (10305 bp), as shown in FIG. 17, having a T-DNA sequence flanked by a right border (with an overdrive element) on the 5′ end and a left border on the 3′ end. The T-DNA sequence is comprised of, in the 5′ to 3′ direction, a constitutive plant promoter (the duplicated mirabilis mosaic virus (dMMV) promoter, a gene of interest which has commercial interest and serves as a selectable marker in tissue culture (the glyphosate-N-acetyltransferase, or GAT variant r9 22-18c5, gene), two 3′ termination regions (the polyubiquitin 3 gene terminator, or UBQ3 3′, and a ribosomal protein gene terminator, or RPG 3′). Downstream of the left border in the non-T-DNA portion of the vector is a visual marker for detecting left border read-through comprised of the Agrobacterium isopentenyl transferase (ipt), or cytokinin autonomy, gene. Following this there is a region that allows plasmid replication and maintenance in Agrobacterium (the RK2 origin of replication, or oriV, and RK2 TrfA region), a kanamycin resistance gene (NPTIII) between the oriV and the RK2 TrfA region for bacterial selection, and an E. coli origin of replication (ori ColE1).

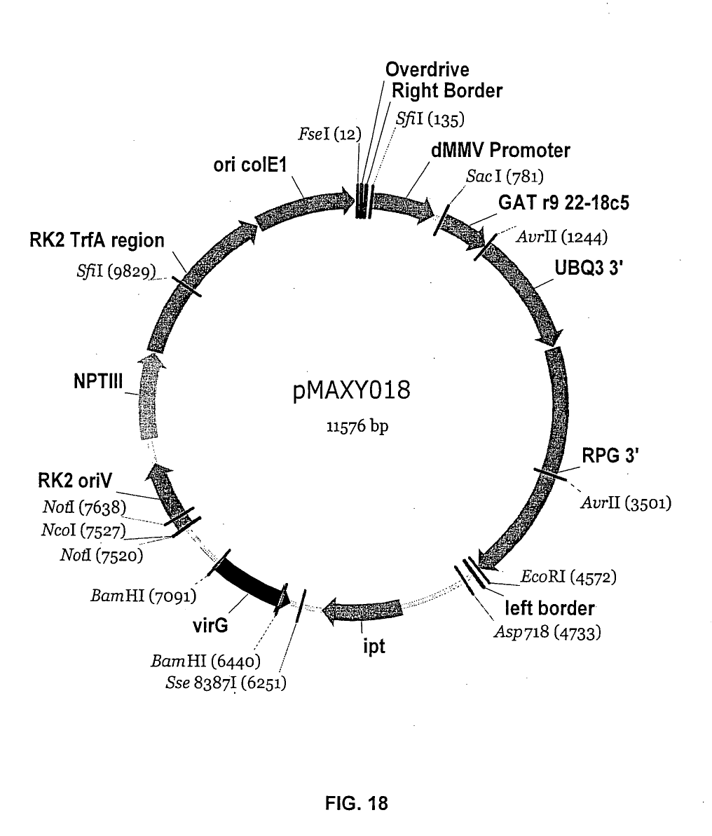

A third vector of the present invention is pMAXY018 (11576 bp), as shown in FIG. 18. This vector is identical to pMAXY017 described above, except that it contains an optional Agrobacterium vir gene, the virG gene, in reverse orientation after the ipt gene.

The present invention is demonstrated in part by the following examples.

### EXAMPLE 1

### Construction of a Binary Vector Backbone Precursor (pMAXY007) of the Present Invention

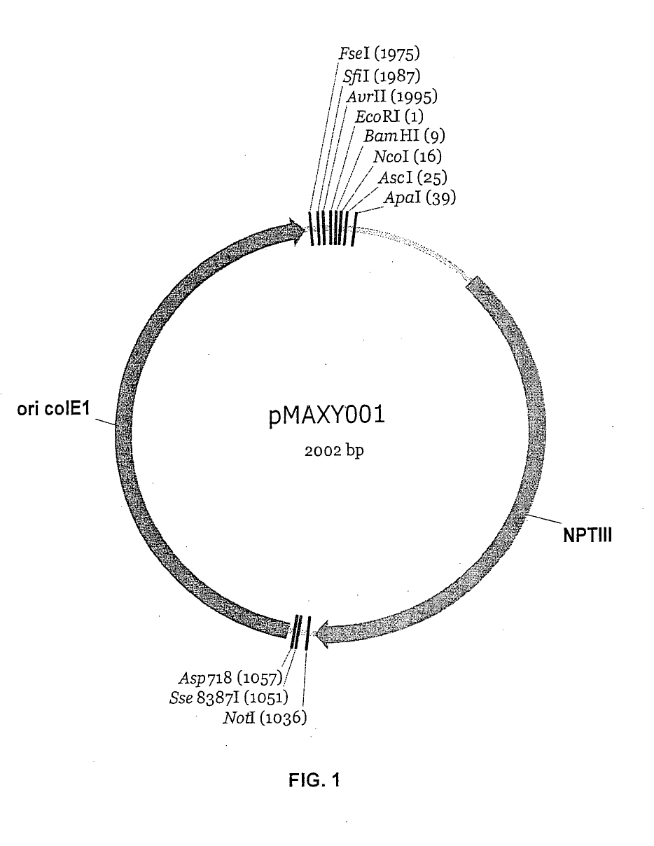

A binary vector precursor was constructed from component elements using the following scheme—Step 1: A ColE1 origin region, which allows plasmid replication and maintenance in E. coli, was PCR-amplified from the vector pBR322 (GenBank #J01749) using primers SEQ ID NO: 5 and SEQ ID NO: 6. These primers introduce Sse8387I and Asp718 sites at the 5′ end of the fragment, and FseI, SfiI, AvrII, EcoRI, and BamHI sites at the 3′ end of the fragment. An NPTIII gene with its associated promoter, which allows antibiotic selection for plasmids in E. coli, was PCR-amplified from the vector pCB301 (Genbank #AF139061) using primers SEQ ID NO: 7 and SEQ ID NO: 8. These primers introduce BamHI, NcoI, AscI, and ApaI sites at the 5′ end of the fragment, and NotI and Sse8387I sites at the 3′ end of the fragment. The ColE1 and NPTIII PCR products were both digested with Sse8387I and BamHI, and then ligated together to create the vector pMAXY001. (See FIG. 1).

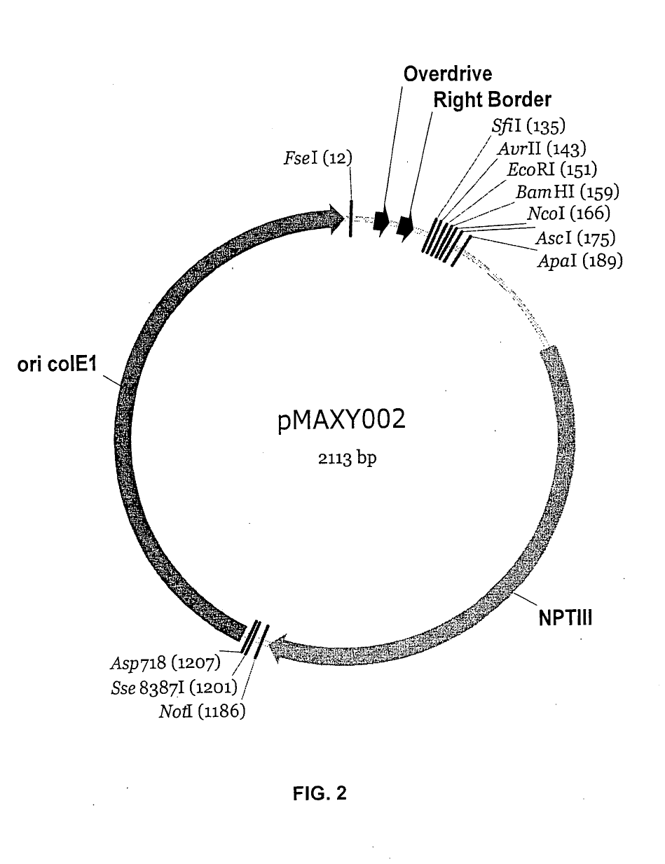

Step 2: A right border and overdrive region, which serves to initiate T-DNA strand synthesis, was PCR-amplified from the plasmid pTiAB3 (Genbank #M63056) using purified Agrobacterium tumefaciens DNA and primers SEQ ID NO: 9 and SEQ ID NO: 10. These primers introduce an FseI site at the 5′ end of the fragment, and an SfiI site at the 3′ end of the fragment. The right border plus overdrive PCR product and the vector pMAXY001 were both digested with FseI and SfiI, and then ligated together to create the vector pMAXY002. (See FIG. 2).

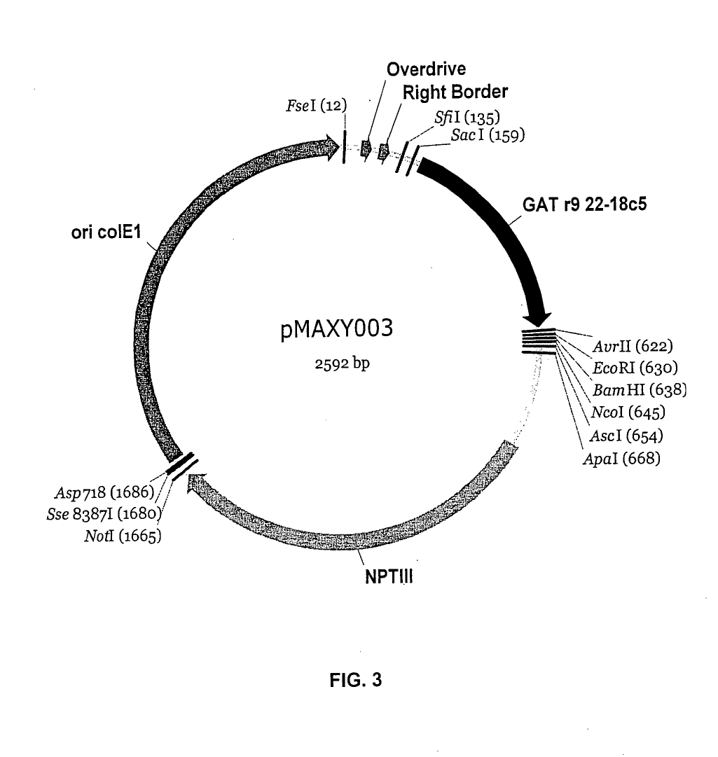

Step 3: A glyphosate-N-acetyltransferase (GAT) gene, which allows selection of transformed plant cells in tissue culture using glyphosate herbicide as the selective agent (Patent App. WO 02/36782 A2, U.S. 2003/0083480 A1), was created by gene synthesis (Stemmer et al., Gene 164:49-53 (1995)) using primers SEQ ID NO: 11-SEQ ID NO: 27. The outside primers introduce SfiI and SacI sites at the 5′ end of the gene, and an AvrII site at the 3′ end of the gene. The PCR-assembled GAT gene (a sequence variant called “r9 22-18c5”) and the vector pMAXY002 were both digested with SfiI and AvrII, and then ligated together to create the vector pMAXY003. (See FIG. 3).

Step 4: A dMMV promoter, which confers high-level constitutive expression in plants, was PCR-amplified from the vector pKM24 (Dey and Maiti, Transgenics 3:61-70, (1999)) using SEQ ID NO: 28 and SEQ ID NO: 29. These primers introduce an SfiI site at the 5′ end of the fragment, and a SacI site at the 3′ end of the fragment. The dMMV promoter PCR product and the vector pMAXY003 were both digested with SfiI and SacI, and then ligated together to create the vector pMAXY004. (See FIG. 4).

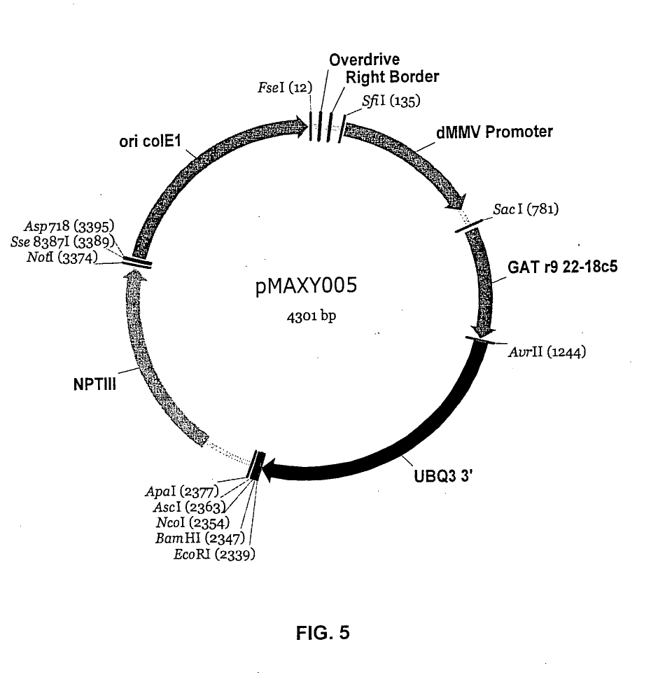

Step 5: A 3′ termination region from the polyubiquitin 3 (UBQ3) gene (Genbank #L05363) was PCR-amplified using purified Arabidopsis thaliana genomic DNA and primers SEQ ID NO: 30 and SEQ ID NO: 31. These primers introduce an AvrII site at the 5′ end of the fragment, and an EcoRI site at the 3′ end of the fragment. The UBQ3 terminator PCR product and the vector pMAXY004 were both digested with AvrII and EcoRI, and then ligated together to create the vector pMAXY005. (See FIG. 5).

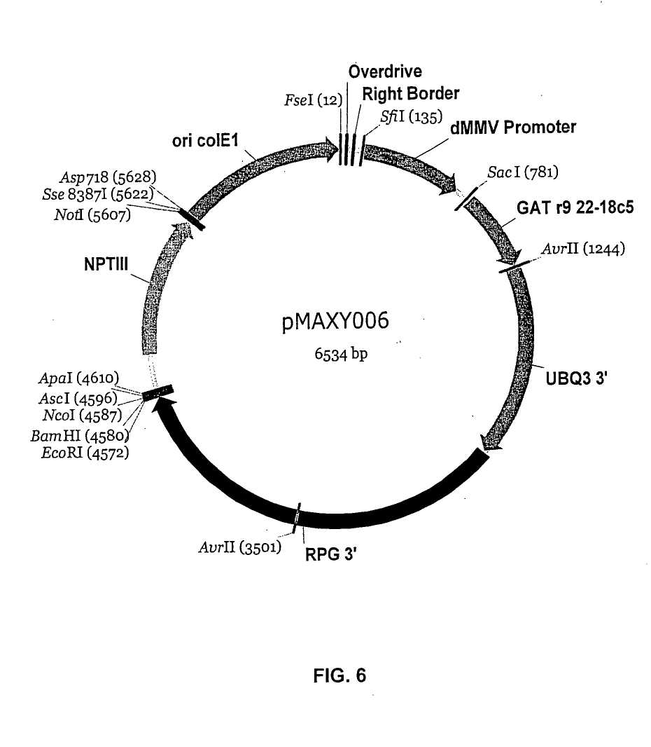

Step 6: A 3′ termination region from the putative ribosomal protein (RPG) gene (Genbank #AP002059), which may provide additional stability to the transcribed GAT mRNA, was PCR-amplified using purified Arabidopsis thaliana genomic DNA and primers SEQ ID NO: 32 and SEQ ID NO: 33. These primers introduce an MfeI site at the 5′ end of the fragment, and an EcoRI site at the 3′ end of the fragment. The RPG terminator PCR product was digested with MfeI (compatible cohesive end with EcoRI) and EcoRI, and the vector pMAXY005 was digested with EcoRI. The two digested DNAs were ligated together to create the vector pMAXY006. (See FIG. 6) The desired orientation of the RPG terminator fragment was determined by restriction digestion analysis and DNA sequencing.

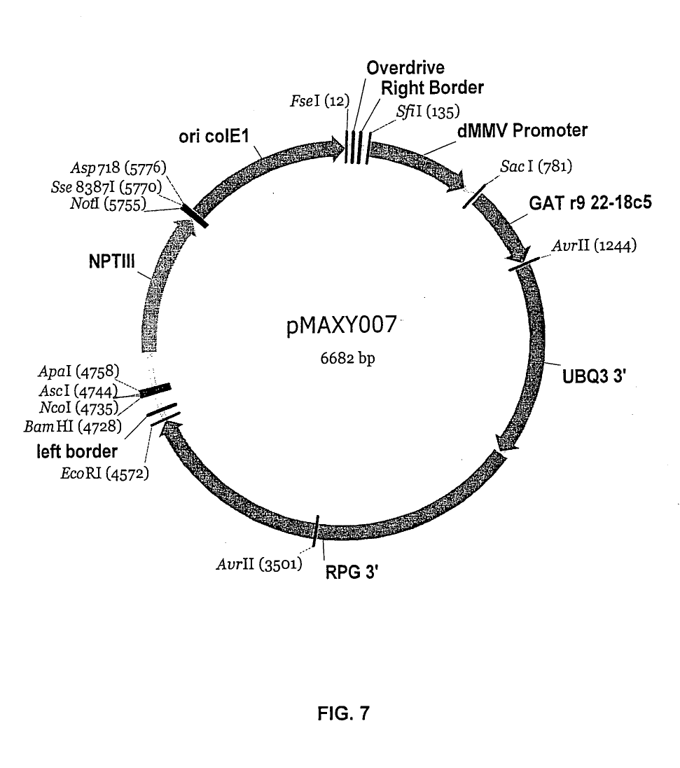

Step 7: A left border region, which serves to terminate T-DNA strand synthesis, was PCR-amplified from the plasmid pTiC58 (Genbank #AE007925) using purified Agrobacterium tumefaciens genomic DNA and primers SEQ ID NO: 34 and SEQ ID NO: 35. These primers introduce an EcoRI site at the 5′ end of the fragment, and a BamHI site at the 3′ end of the fragment. The left border PCR product and the vector pMAXY006 were both digested with EcoRI and BamHI, and then ligated together to create the vector pMAXY007. (See FIG. 7).

The vector pMAXY007 is the base binary vector precursor into which elements of the present invention are inserted. It should be realized that this base vector is provided for illustrative purposes only, and that other commonly used plant binary vectors could be modified by one skilled in the art to incorporate elements of the present invention.

### EXAMPLE 2

### Insertion of a GFP Visual Marker Expression Cassette into the Binary Backbone Precursor

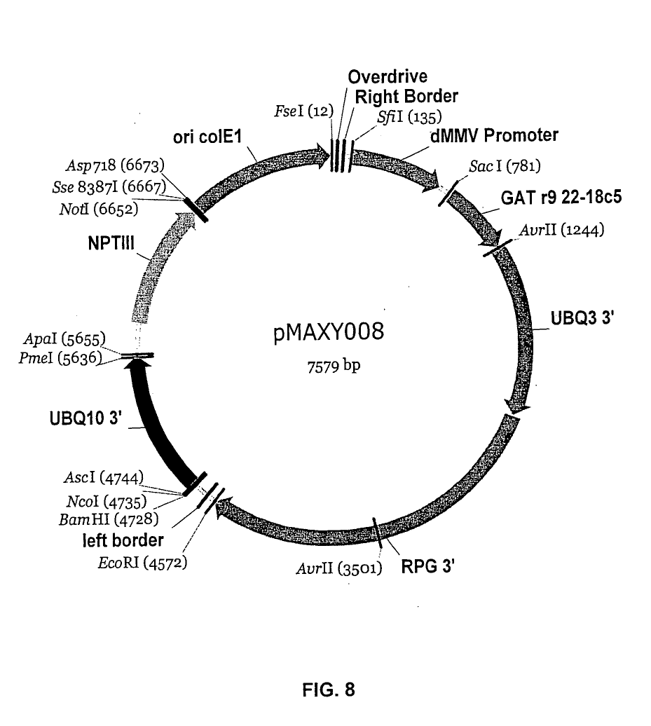

Step 1: A 3′ termination region from the polyubiquitin 10 (UBQ10) gene (Genbank #NC 003075) was PCR-amplified using purified Arabidopsis thaliana genomic DNA and primers SEQ ID NO: 36 and SEQ ID NO: 37. These primers introduce an AscI site at the 5′ end of the fragment, and PmeI and ApaI sites at the 3′ end of the fragment. The UBQ10 terminator PCR product and the vector pMAXY007 were both digested with AscI and ApaI, and then ligated together to create the vector pMAXY008. (See FIG. 8).

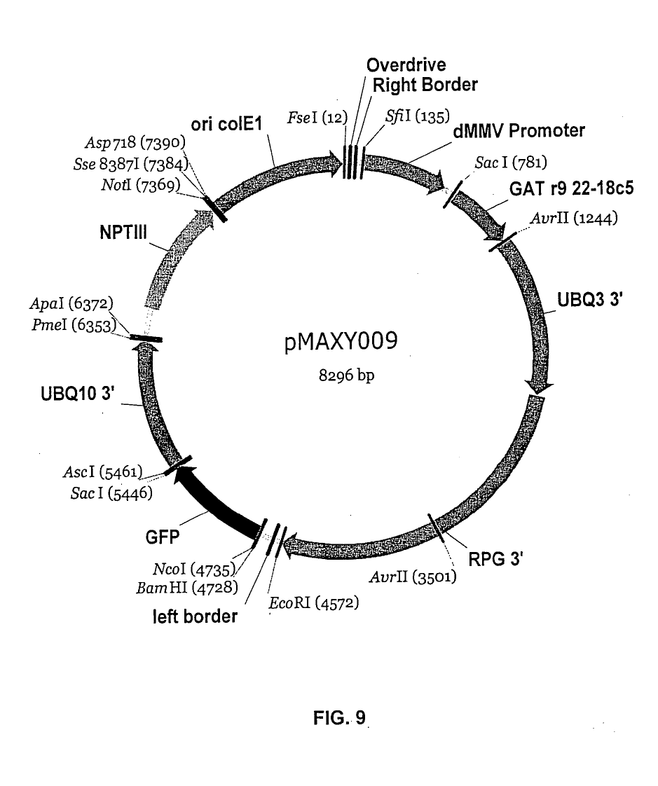

Step 2: A cycle 3 green fluorescent protein (GFP) gene (Crameri et al., Nat. Biotechnol. 14:315-319 (1996)) was created by gene synthesis (Stemmer et al., Gene 164:49-53 (1995)) using primers SEQ ID NO: 38-SEQ ID NO: 61. The primers were designed to eliminate a cryptic plant intron within the gene. In addition, the outside primers introduce an NcoI site at the 5′ end of the fragment, and an AscI site at the 3′ end of fragment. The PCR-assembled GFP gene and the vector pMAXY008 were both digested with NcoI and AscI, and then ligated together to create the vector pMAXY009. (See FIG. 9).

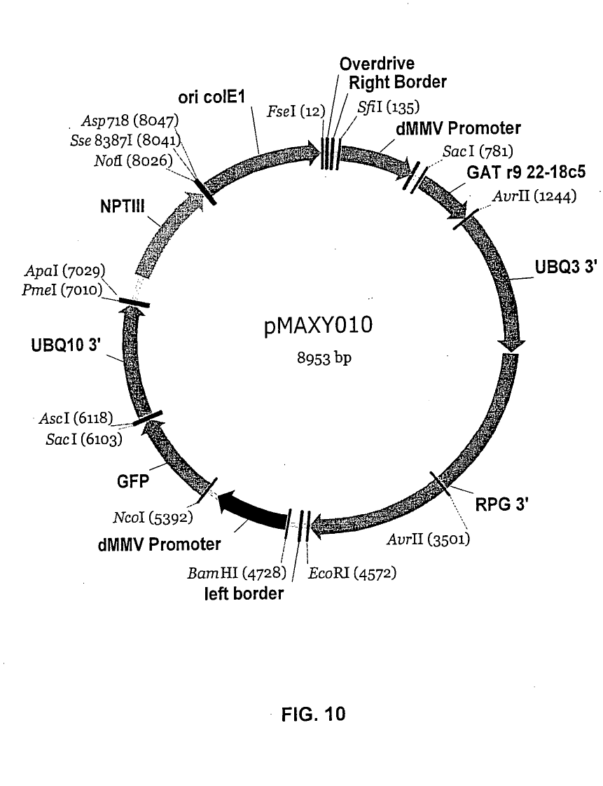

Step 3: A dMMV promoter was PCR-amplified from pKM24 (Dey and Maiti, Transgenics 3:61-70 (1999)) using primers SEQ ID NO: 62 and SEQ ID NO: 63. These primers introduce a BamHI site at the 5′ end of the fragment, and an NcoI site at the 3′ end of fragment. The dMMV promoter PCR product and the vector pMAXY009 were both digested with BamHI and NcoI, and then ligated together to create the vector pMAXY010. (See FIG. 10).

The assembled cassette will express the GFP visual marker gene in plant cells using the constitutive dMMV promoter and the UBQ10 3′ terminator. Note that other fluorescent protein or alternative visual marker genes could be substituted for the GFP gene using a similar PCR and restriction digest approach. Alternative plant promoter and 3′ terminator elements could also be used in place of those described above.

### EXAMPLE 3

### Insertion of a Cytokinin Autonomy Visual Marker Gene into the Binary Backbone Precursor

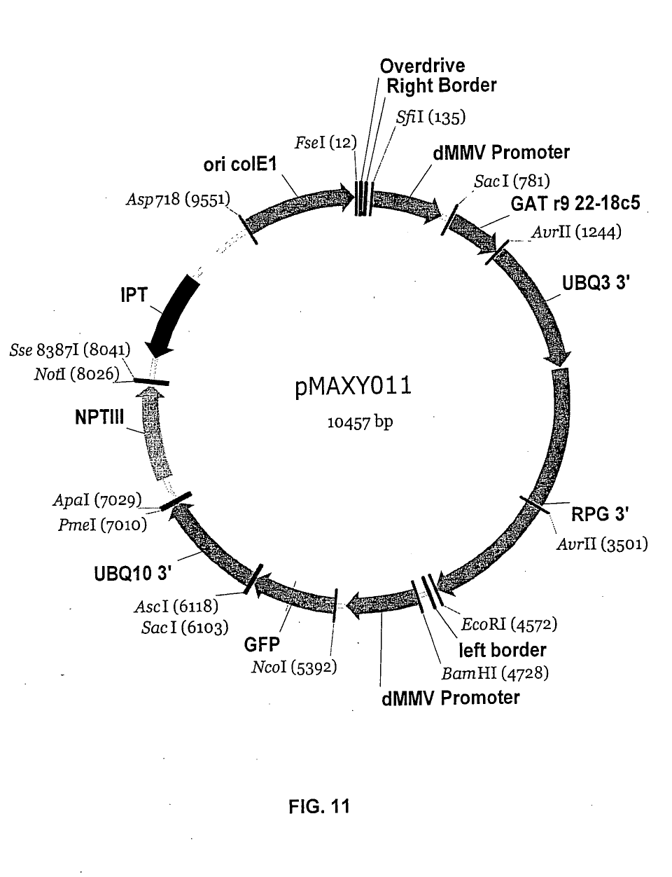

The isopentenyl transferase (IPT) gene with its associated promoter and 3′ terminator was PCR-amplified from the plasmid pTiC58 (Genbank #AE009419) using purified Agrobacterium tumefaciens DNA and primers SEQ ID NO: 64 and SEQ ID NO: 65. These primers introduce an Asp718 site at the 5′ end of the fragment, and an Sse8387I site at the 3′ end of the fragment. The IPT gene PCR product and the vector pMAXY010 were both digested with Asp718 and Sse8387I, and then ligated together to create the vector pMAXY011. (See FIG. 11). In this vector design, the IPT gene is placed in an opposite orientation to the direction of the GAT and GFP expression cassettes. In an alternative design, the IPT gene could be placed in the same orientation as these other cassettes. Moreover, other cytokinin autonomy genes could be substituted for the A. tumefaciens IPT gene using a similar PCR and restriction digest approach.

### EXAMPLE 4

### Insertion of a Vir Gene into the Binary Backbone Precursor

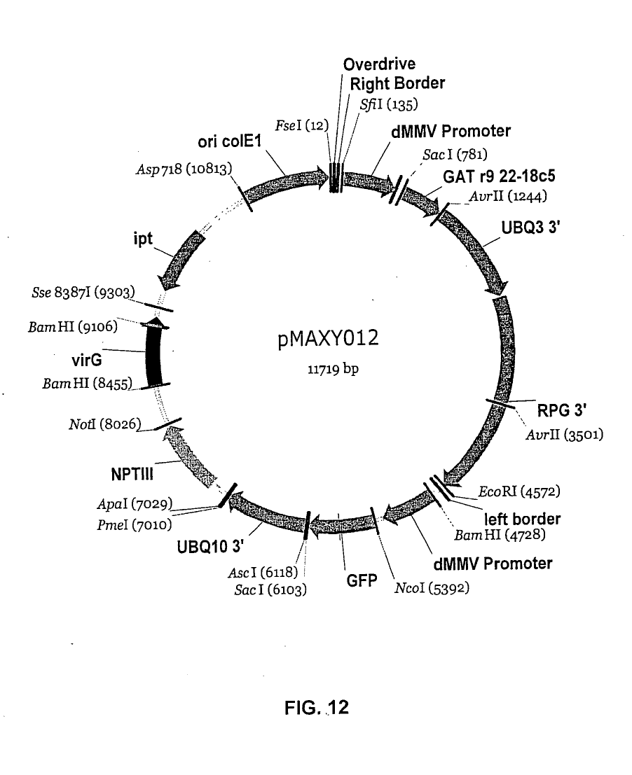

A virG gene with its associated promoter and 3′ terminator was PCR-amplified from the plasmid pTiC58 (Genbank #AE009436) using purified Agrobacterium tumefaciens DNA and primers SEQ ID NO: 66 and SEQ ID NO: 67. These primers introduce NcoI and NotI sites at the 5′ end of the fragment, and an Sse8387I site at the 3′ end of the fragment. The VirG gene PCR product and the vector pMAXY011 were both digested with NotI and Sse8387I, and then ligated together to create the vector MAXY012. (See FIG. 12). Note that other vir genes or vir regions could be substituted for the A. tumefaciens virG gene using a similar PCR and restriction digest approach.

### EXAMPLE 5

### Insertion of an Agrobacterium Origin of Replication into the Binary Backbone Precursor

A DNA fragment from the Pseudomonas PVS1 plasmid, which contains an origin of replication and the RepA and StaA genes (Itoh and Haas, Gene 36:27-36 (1985)), was PCR-amplified from the vector pCAMBIA2301 (Genbank #AF234316) using primers SEQ ID NO: 68 and SEQ ID NO: 69. These primers introduce a PmeI site at the 5′ end of the fragment, and an ApaI site at the 3′ end of the fragment. The PVS1 origin PCR product and the vector pMAXY012 were both digested with PmeI and ApaI, and then ligated together to create the vector pMAXY013. (See FIG. 13). Note that other origins of replication could be substituted for the PVS1 ori region using a similar PCR and restriction digest approach.

### EXAMPLE 6

### Insertion of Extra Left Border Repeats into the Binary Backbone

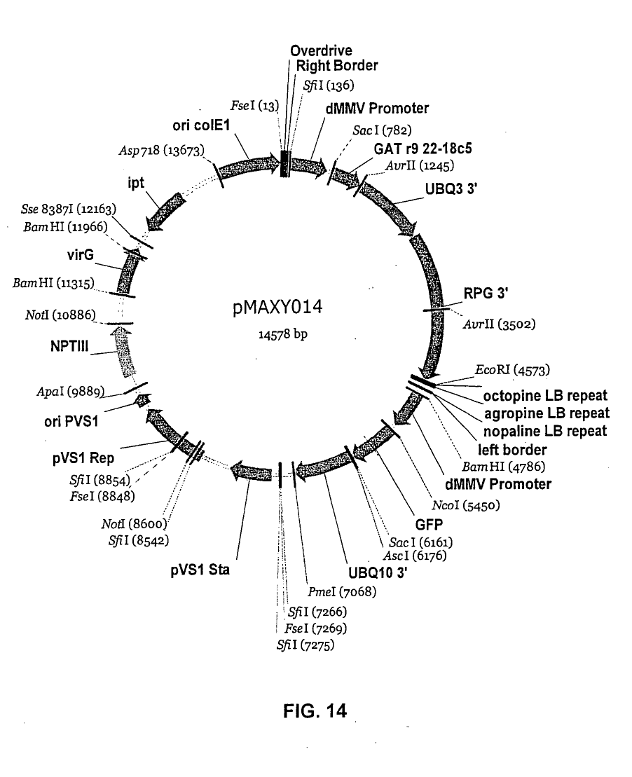

A double-stranded oligonucleotide containing extra left border repeats was created by annealing SEQ ID NO: 70 and SEQ ID NO: 71 to each other. This oligonucleotide contains an octopine-type left border repeat and an agropine-type left border repeat, although an experimenter could design oligonucleotides that contain multiple copies of a single repeat-type if desired. The vector backbones of EXAMPLES 1-5 already contain a nopaline-type left border repeat. The two single-stranded oligonucleotides (SEQ ID NO: 70, NO: 71) were designed such that upon annealing to each other, a 4-nucleotide single-stranded region is created at both ends of the molecule. These “sticky ends” are compatible for ligation into the EcoRI site of the vectors in EXAMPLES 1-5. Because the double-stranded oligonucleotides can also ligate to themselves, it is possible to have more than one copy of the annealed oligonucleotide, and thus more left border repeats, inserted into the vector backbone. The desired number and orientation of inserted repeats can be determined and selected by sequencing individual vectors from the ligation reaction. As an example, the insertion of one annealed oligonucleotide in one orientation into the EcoRI site of pMAXY013 was carried out to create the vector pMAXY014. (See FIG. 14).

### EXAMPLE 7

### Insertion of a Cytokinin Autonomy Visual Marker Gene and a Vir Gene into the Binary Backbone at Alternative Positions

Various elements disclosed herein can be inserted into various positions of a binary vector backbone as long as the functional properties of these elements are not disrupted during the cloning process. These different vector configurations are considered to be within the realm of the present invention. As an example of this modularity, an alternative vector configuration was designed using the following scheme:

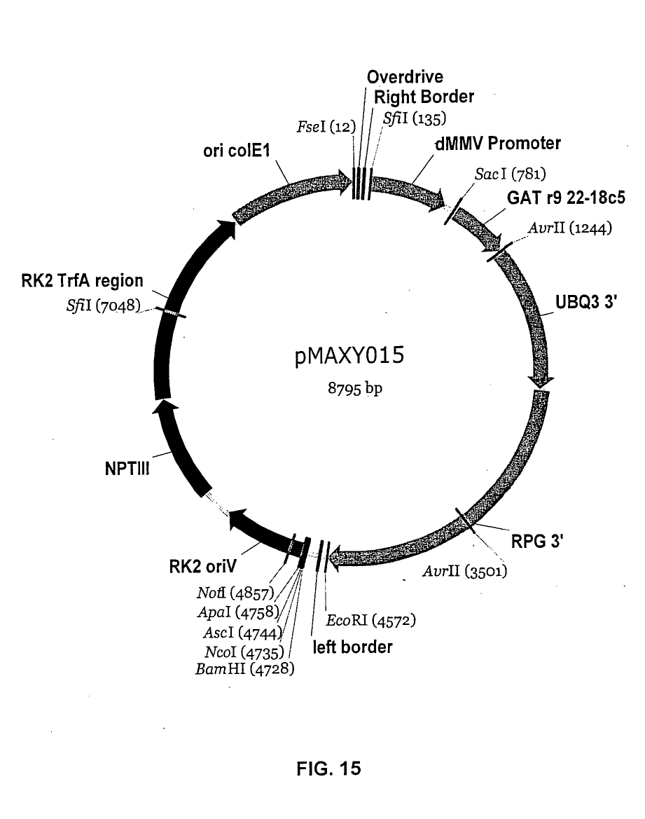

Step 1: A DNA fragment from the plasmid pRK2, which contains the oriV region and TrfA gene for plasmid replication and maintenance in Agrobacterium, and a fragment containing the NPTIII gene were PCR-amplified as a single product from the vector pCB301 (Genbank #AF139061) using primers SEQ ID NO: 72 and SEQ ID NO: 73. These primers introduce an ApaI site at the 5′ end of the PCR product and a BsrGI site at the 3′ end of the PCR product. The oriV-NPTIII-TrfA PCR product was digested with BsrGI (compatible cohesive end with Asp718) and ApaI, and the vector pMAXY007 was digested with ApaI and Asp718. The two digested DNAs were ligated together to create the vector pMAXY015. (See FIG. 15).

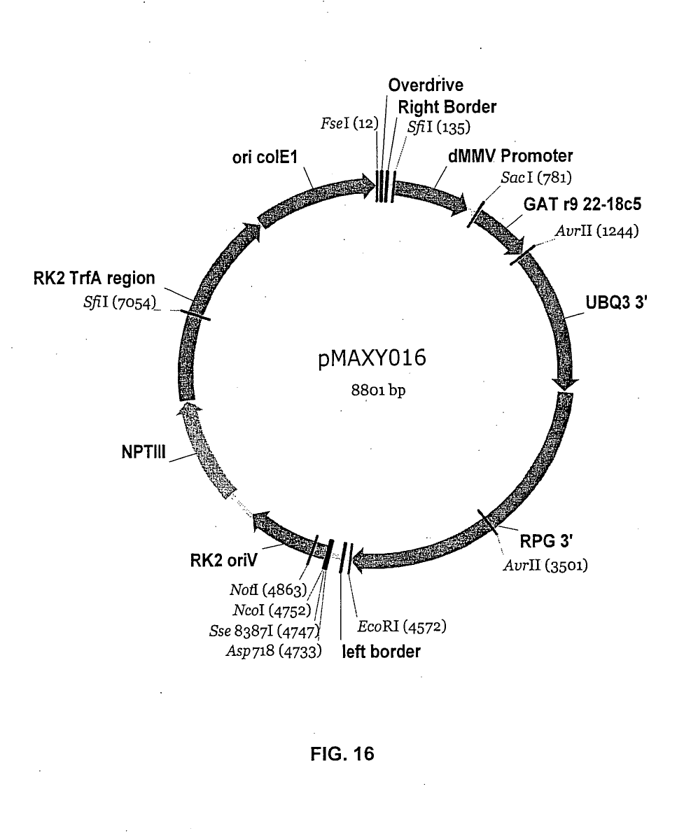

Step 2: A double-stranded oligonucleotide was created by annealing primers SEQ ID NO: 74 and SEQ ID NO: 75. This oligonucleotide forms an adapter with internal Asp718, Sse8387I, and NcoI sites, and has external “sticky ends” that are compatible with but eliminate BamHI and ApaI sites upon ligation. The vector pMAXY015 was digested with BamHI and ApaI, and then ligated to the annealed primers to create the vector pMAXY016. (See FIG. 16).

Step 3: An IPT gene with its associated promoter and 3′ terminator was PCR-amplified from the plasmid pTiC58 (Genbank #AE009419) using purified Agrobacterium tumefaciens DNA and primers SEQ ID NO: 64 and SEQ ID NO: 65. These primers introduce an Asp718 site at the 5′ end of the fragment, and an Sse8387I site at the 3′ end of the fragment. The IPT gene PCR product and the vector pMAXY016 were both digested with Asp718 and Sse8387I, and then ligated together to create pMAXY017. (See FIG. 17). In this vector design, the IPT gene is placed in the same orientation as the direction of the GAT expression cassette. In an alternative design, the IPT gene could be placed in an opposite orientation to the internal T-DNA expression cassette.

Step 4: A virG gene with its associated promoter and 3′ terminator was PCR-amplified from the plasmid pTiC58 (Genbank #AE009436) using purified Agrobacterium tumefaciens DNA and primers SEQ ID NO: 66 and SEQ ID NO: 67. These primers introduce NcoI and NotI sites at the 5′ end of the fragment, and an Sse8387I site at the 3′ end of the fragment. The virG gene PCR product and the vector pMAXY017 were both digested with NcoI and Sse8387I, and then ligated to create the vector pMAXY018. (See FIG. 18).

### EXAMPLE 8

### Transforming Plant Cells with the Binary Vectors of Examples 5, 6 and 7

The binary vectors were transformed into competent Agrobacterium tumefaciens strain EHA105 cells by electroporation (McCormac et al., Mol Biotechnol. 9:155-159 (1998)). Axillary buds of Nicotiana tabacum L. Xanthi were sub-cultured on half-strength Murashige and Skoog (MS) media with sucrose (1.5%) and Gelrite (0.3%) under 16-h light (35-42 μEinsteins m⁻²s⁻¹, cool white fluorescent lamps) at 24° C. every 2-3 weeks. Young leaves were excised from plants after 2-3 weeks subculture and then cut into 3×3 mm segments. A. tumefaciens cells containing the binary vectors of interest were inoculated into LB medium+100 μg/ml kanamycin and grown overnight to a density of A600=1.0. Cells were pelleted at 4,000 rpm for 5 minutes and resuspended in 3 volumes of liquid co-cultivation medium composed of MS medium (pH 5.2) with 2 mg/L N6-benzyladenine (BA), 1% glucose, and 400 μM acetosyringone. The leaf pieces were then fully submerged in 20 ml of A. tumefaciens suspension in 100×25 mm Petri dishes for 30 minutes, blotted on solid co-cultivation medium (0.3% Gelrite), and incubated as described above. After 3 days of co-cultivation, 20-30 segments were transferred to basal shoot induction (BSI) medium composed of MS solid medium (pH5.7) with 2 mg/L BA, 3% sucrose, 0.3% Gelrite, 50-400 μM glyphosate, and 400 μg/ml Timentin. Transformed plant material resistant to the glyphosate selective agent could be observed after 2-3 weeks. At 3-4 weeks, organized shoots with expanding leaves were clearly evident.

### EXAMPLE 9

### Detecting Read-Through into the Non-T-DNA Cytokinin Autonomy Gene in the Transformants of Example 8

Transformed plant cells and tissues on petri plates were examined for phenotypic changes associated with cytokinin over-production. The manifestation of these phenotypes is highly dependent on the transformation protocol used and the concentrations of exogenous hormones (auxin and cytokinin) that are supplied in the media. In some cases, as with the protocol and media described in EXAMPLE 8, the vast majority of the transformed material rapidly forms normal shoots, which can be easily rooted in soil and propagated as phenotypically normal plants. The IPT-expressing material does not develop into normal shoots, and therefore this undifferentiated or phenotypically abnormal material is selected against and easily avoided using this protocol. In contrast, if the BSI media of EXAMPLE 8 is modified such that it contains no exogenous BA, then any transformed material expressing the cytokinin autonomy gene can be clearly distinguished. After a period of several weeks, the IPT-containing material forms shooty tumors that are very characteristic of cytokinin over-production. This abnormal shoot tissue does not readily produce roots, and any regenerated plantlets that are formed tend to have altered morphology. Thus, an experimenter can manipulate the transformation protocol and media composition to facilitate easy detection of cytokinin autonomy gene expression, or to preferentially yield transformed plants that do not contain the cytokinin autonomy gene.

### EXAMPLE 10

### Detecting Read-Through into the Non-T-DNA Fluorescent Protein Marker Cassette in the Transformants of Example 8

Transformed plant cells and tissues on petri plates were examined with a Leica model MZ-FLIII microscope equipped with a Chroma Technology Corp., Sapphire/UV GFP (D395/40x, 425D CLP, D510/40m), main ID 31043 filter set and a Leica model 106z 100w Hg light source. Cells expressing the GFP protein as a result of left border read-through were identified visually by light emission that appears bright green to the human eye. Surrounding tissue that does not express the GFP visual marker gene appears clear, red (due to chlorophyll fluorescence), or possibly another color, but not fluorescent green due to light emission from the GFP protein. Alternatively, transformed tissue was examined using a UV light box (VWR Scientific Transilluminator, Model LM-20E), an LED flashlight (Emissive Energy Corp., Inova X5 LED Floodlight, model X5MT-UV), or a similar device that provides an appropriate excitation frequency for the GFP protein. Using these or similar methods, the experimenter is able to separate and propagate transgenic material that does not show GFP fluorescence and, therefore, is less likely to contain non-T-DNA sequences integrated into the genome. Microscope filter sets and excitation light sources can be altered for the detection of fluorescent proteins other than GFP.

### EXAMPLE 11

### Data to Support the Use of the IPT Gene as a Visual Marker to Identify Transgenic Plant Material Containing Read-Through (Non-T-DNA) Sequences and/or to Produce a Transgenic Plant Substantially Free of Non-T-DNA

| | # of events with “shooty” | PCR confirmation of IPT gene |
|---|---|---|
| | phenotype/ total # of explants | in events with “shooty” |
| Vector | transformed | phenotype |
| Agro. only | 0/180 (0.0%) | N.A. |
| 5014 | 14/180 (7.8%) | 14/14 (100%) |
| 3085 | 0/180 (0.0%) | N.A. |

Background: Two binary vectors were created to validate the use of the IPT gene as a visual marker for identifying transfer of vector backbone (non-T-DNA) sequences into transformed plant material. The vector pMAXY5014 is composed of: Right Border-dMMV promoter-glyphosate acetyltransferase gene-ubiquitin 3 gene terminator-Left Border and then 810 bp to the IPT gene in the vector backbone. The vector pMAXY3085 is identical to pMAXY5014, except that the IPT gene was disrupted by removal of an internal segment of the gene. These binary vectors were introduced into Agrobacterium cells by electroporation and then used to transform tobacco leaf explants as described in Example 9. After 6-8 weeks in culture, the explants were examined for the presence of a “shooty” teratoma outgrowth that is characteristic of cytokinin overproduction due to expression of the IPT gene. The vector (5014) containing a functional cytokinin autonomy gene produced “shoots” with the characteristic teratoma phenotype at a low but detectable frequency (7.8%), whereas infection with a vector (3085) containing a non-functional IPT gene or use of Agrobacterium cells lacking a binary vector never produced “shoots” of this type. DNA was extracted from the 14 teratoma “shoots” and 2 non-transformed shoots, PCR analysis was conducted with primers designed to amplify the IPT gene, and the resulting products were evaluated by agarose gel electrophoresis. All 14 of the teratoma DNA samples yielded a band of the expected size for the IPT gene, whereas the negative control samples gave no PCR product. Thus, there was a perfect correlation between the presence of the IPT gene (non-T-DNA sequence) by PCR analysis, the appearance of a “shooty” teratoma phenotype, and the ability to visually select for transgenic events that are substantially free of non-T-DNA.

### EXAMPLE 12

### Data to Support the Use of the GFP Gene as a Visual Marker to Identify Transgenic Plant Material Containing Read-Through (Non-T-DNA) Sequences and/or to Produce a Transgenic Plant Substantially Free of Non-T-DNA

Background: Two binary vectors were created to validate the use of the GFP gene as a visual marker for identifying transfer of vector backbone (non-T-DNA) sequences into transformed plant material. The vector pMAXY5701 is composed of: Right Border-dMMV promoter-glyphosate acetyltransferase gene-ubiquitin 3 gene terminator-three Left Border elements and then dPCSV promoter-cycle 3 GFP gene-ubiquitin 10 gene terminator in the vector backbone. The vector pMAXY5702 is composed of: Right Border-dMMV promoter-Cryl insecticidal protein gene-ubiquitin 14 gene terminator-SVBV promoter-glyphosate acetyltransferase gene-ubiquitin 3 gene terminator-three Left Border elements and then dPCSV promoter 3 GFP gene-ubiquitin 10 gene terminator in the vector backbone. These binary vectors were introduced into Agrobacterium cells by electroporation and then used to transform tobacco leaf explants as described in Example 8. Transgenic tobacco plants were produced with glyphosate selection as described in Science 304 (5674):1151-1154, 2004. The plants were examined at two stages (in tissue culture and in soil) for visible GFP fluorescence using a hand-held UV light source as described in Example 10. DNA was extracted from thirty-nine 5701 events and twenty-seven 5702 that showed no visible green fluorescence. PCR analysis was conducted with primers designed to amplify the GFP gene, and the resulting products were evaluated by agarose gel electrophoresis. All of the events for 5702 were negative for the GFP gene, whereas 85% of the events for 5701 were PCR-negative. The GFP-positive samples can be explained as a false amplification signal in the PCR test, the presence of a GFP fragment in the genome that is nonfunctional, or the presence of an intact GFP gene in the genome that is expressed below the level of visual detection with a UV light source. Southern blot analysis was also performed on genomic DNA extracted from a small subset of the 5701 and 5702 events. Consistent with the PCR analysis, the same events were found to be PCR-positive or PCR-negative using this alternative molecular test. Thus, there was a good correlation between the absence of the GFP gene (non-T-DNA sequence) by visual inspection, the lack of an integrated GFP gene by PCR and Southern blot analyses, and the ability to visually select for transgenic events that are substantially free of non-T-DNA.

What is claimed is:

1. An isolated T-DNA vector comprising: i) a right border, a left border and a T-DNA sequence therebetween, said T-DNA sequence therebetween lacking a functional cytokinin autonomy gene; and ii) a non-T-DNA sequence beyond said left border, said non-T-DNA sequence comprising an Agrobacterium origin of replication and a first visual marker gene, said first visual marker gene being a cytokinin autonomy gene.

2. The isolated T-DNA vector of claim 1, wherein the cytokinin autonomy gene in the non-T-DNA sequence is from Agrobacterium tumefaciens.

3. The isolated T-DNA vector of claim 1, wherein the Agrobacterium origin of replication is derived from the ori region of the Pseudomonas PVS1 plasmid or the ori region of the RK2 broad-host range plasmids.

4. The isolated T-DNA of claim 1, wherein the left border has been modified to comprise more than one left border sequence.

5. The isolated T-DNA vector of claim 1, wherein the non-T-DNA sequence further comprises a second visual marker gene.

6. The isolated T-DNA vector of claim 5, wherein said second visual marker gene is a gene encoding a fluorescent protein, or a gene involved in the synthesis and accumulation of an anthocyanin, a carotenoid or an indigo pigment.

7. The isolated T-DNA vector of claim 6, wherein the fluorescent protein is a green fluorescent protein.

8. The isolated T-DNA vector of claim 7, wherein the green fluorescent protein is cycle 3 GFP.

9. The isolated T-DNA vector of claim 8, wherein the green fluorescent protein is under operative control of a constitutive plant promoter.

10. The method of claim 1, wherein said non-T-DNA sequence further comprises a polynucleotide encoding one or more Agrobacterium vir proteins.

11. A method for producing a transgenic plant containing a polynucleotide of interest but being substantially free of non-T-DNA, the method comprising:
(a) introducing into a plurality of plant cells a T-DNA vector comprising:
(i) a right border, a left border and a T-DNA sequence therebetween, said T-DNA sequence therebetween lacking a functional cytokinin autonomy gene; and
(ii) a non-T-DNA sequence beyond said left border, said non-T-DNA sequence comprising an Agrobacterium origin of replication and a first visual marker gene, said first visual marker gene being a cytokinin autonomy gene;
(b) selecting a plant cell which expresses the T-DNA sequence and does not visually manifest abnormal cell proliferation, accelerated or deeper “greening,” delayed senescence, or the formation of “shooty” tumors associated with read-through and expression of cytokinin autonomy gene from the non-T-DNA sequence; and
(c) regenerating a transgenic plant from the selected plant cell.

12. The method of claim 11, wherein the cytokinin autonomy gene in the non-T-DNA sequence is from Agrobacterium tumefaciens.

13. The method of claim 11, wherein the Agrobacterium origin of replication is derived from the ori region of the Pseudomonas PVS1 plasmid or the ori region of the RK2 broad-host range plasmids.

14. The method of claim 11, wherein the left border has been modified to comprise from more than one left border sequence.

15. The method of claim 11, wherein the non-T-DNA sequence further comprises a second visual marker gene.

16. The method of claim 11, wherein said second visual marker gene is a gene encoding a fluorescent protein, or a gene involved in the synthesis and accumulation of an anthocyanin, a carotenoid or an indigo pigment.

17. The method of claim 11, wherein said non-T-DNA sequence further comprises a polynucleotide encoding one or more Agrobacterium vir proteins.

18. A method for producing a transgenic plant containing a polynucleotide of interest but being substantially free of non-T-DNA, the method comprising:
(a) transforming a plurality of plant cells with a T-DNA vector comprising:
(i) a right border, a left border and a T-DNA sequence therebetween, the T-DNA sequence therebetween lacking a functional cytokinin autonomy gene; and
(ii) a non-T-DNA sequence beyond the left border, the non-T-DNA sequence comprising an Agrobacterium origin of replication, a first visual marker gene, and a second visual marker gene, wherein the first visual marker gene is a cytokinin autonomy gene, and the second visual marker gene encodes a visually detectable fluorescent protein;
(b) selecting a transformed plant cell and its progeny that do not have visible fluorescence upon illumination with an appropriate light source, wherein fluorescence is associated with read-through and expression of the fluorescent protein gene from the non-T-DNA sequence; and
(c) regenerating a transgenic plant from the selected plant cell.

19. A method for producing a transgenic plant containing a polynucleotide of interest but being substantially free of non-T-DNA, the method comprising:
(a) transforming a plurality of plant cells with a T-DNA vector comprising:
(i) a right border, a left border and a T-DNA sequence therebetween, the T-DNA sequence therebetween containing a polynucleotide of interest and lacking a functional cytokinin autonomy gene; and
(ii) a non-T-DNA sequence beyond the left border, the non-T-DNA sequence comprising an Agrobacterium origin of replication, a first visual marker gene, and a second visual marker gene, wherein the first visual marker gene is a cytokinin autonomy gene, and the second visual marker gene encodes a protein that directs the synthesis of a visually detectable chemical compound;
(b) selecting a transformed plant cell and its progeny that do not have visible accumulation of the chemical compound, which is associated with read-through and expression of the pigment synthesis gene from the non-T-DNA sequence; and
(c) regenerating a transgenic plant from the selected plant cell.


SEQUENCE LISTING (75 sequences)

SEQ ID NO: 1 - DNA, 22 bp, Agrobacterium tumefaciens
ggcaggatatataccgttgtaa

SEQ ID NO: 2 - DNA, 25 bp, Agrobacterium tumefaciens
tgacaggatatattggcgggtaaac

SEQ ID NO: 3 - DNA, 22 bp, Agrobacterium tumefaciens
ggcaggatatattcaattgtaa

SEQ ID NO: 4 - DNA, 25 bp, Agrobacterium tumefaciens
tggcaggatatattgtggtgtaaac

SEQ ID NO: 5 - DNA, 71 bp, Artificial Sequence
  oligonucleotide
gaaacgcctgcaggaacggtacctagtgcgctcttccgcttcctcgctcactgactcgctgcgctcggtcg

SEQ ID NO: 6 - DNA, 91 bp, Artificial Sequence
  oligonucleotide
gttaggatcccagaattcttcctaggacggcctgataggccaaggccggccacgcttcatttttaatttaaaaggatctaggtgaagatcc

SEQ ID NO: 7 - DNA, 86 bp, Artificial Sequence
  oligonucleotide
ttctatcggatccgccatggatggcgcgccagtgggcccataattgtggtttcaaaatcggctccgtcgatactatgttatacgcc

SEQ ID NO: 8 - DNA, 83 bp, Artificial Sequence
  oligonucleotide
catctaacctgcaggatggcggccgcatctaggtactaaaacaattcatccagtaaaatataatattttattttctcccaatc

SEQ ID NO: 9 - DNA, 40 bp, Artificial Sequence
  oligonucleotide
acgtcacactggccggcctggaatcgcttctacaaaactc

SEQ ID NO: 10 - DNA, 43 bp, Artificial Sequence
  oligonucleotide
ttttttggcctgataggccgatgttttggtgctggctgacagg

SEQ ID NO: 11 - DNA, 58 bp, Artificial Sequence
  oligonucleotide
aaaaaaaggcctatcaggccgcaggtcgacttaagagctcaaaaaaaaaaaaaacatg

SEQ ID NO: 12 - DNA, 60 bp, Artificial Sequence
  oligonucleotide
attgaagtaaaaccaatcaatgcggaagatacctatgatctgcgtcaccgcgtgctgcgg

SEQ ID NO: 13 - DNA, 60 bp, Artificial Sequence
  oligonucleotide
ccgaaccagccgatcgaagcatgcatgttcgaaagcgatctgacccgcggtgccttccat

SEQ ID NO: 14 - DNA, 60 bp, Artificial Sequence
  oligonucleotide
ctgggcggtttctacggaggtaagctgatcagcgttgcgtcgttccatcaagctgagcat

SEQ ID NO: 15 - DNA, 60 bp, Artificial Sequence
  oligonucleotide
agcgagttgcagggacagaagcagtaccagcttcgaggtgttgcaaccctggaaggctat

SEQ ID NO: 16 - DNA, 60 bp, Artificial Sequence
  oligonucleotide
cgcgaacaaaaagcgggtaccagcttggttaaacacgctgaagaaattctgcgtaaacgc

SEQ ID NO: 17 - DNA, 60 bp, Artificial Sequence
  oligonucleotide
ggtgttgatctcctgtggtgcaatgctcgtacgtcggcttcgggctactaccgcaaactt

SEQ ID NO: 18 - DNA, 60 bp, Artificial Sequence
  oligonucleotide
gggtttagcgagcagggtgaagtttttgataccccgccggtaggaccgcacattctgatg

SEQ ID NO: 19 - DNA, 35 bp, Artificial Sequence
  oligonucleotide
tataaaagaatcacctaaggcgccctaggtttttt

SEQ ID NO: 20 - DNA, 60 bp, Artificial Sequence
  oligonucleotide
atcttccgcattgattggttttacttcaatcatgttttttttttttttgagctcttaagt

SEQ ID NO: 21 - DNA, 60 bp, Artificial Sequence
  oligonucleotide
gaacatgcatgcttcgatcggctggttcggccgcagcacgcggtgacgcagatcataggt

SEQ ID NO: 22 - DNA, 60 bp, Artificial Sequence
  oligonucleotide
gatcagcttacctccgtagaaaccgcccagatggaaggcaccgcgggtcagatcgctttc

SEQ ID NO: 23 - DNA, 60 bp, Artificial Sequence
  oligonucleotide
ctggtactgcttctgtccctgcaactcgctatgctcagcttgatggaacgacgcaacgct

SEQ ID NO: 24 - DNA, 60 bp, Artificial Sequence
  oligonucleotide
aaccaagctggtacccgctttttgttcgcgatagccttccagggttgcaacacctcgaag

SEQ ID NO: 25 - DNA, 60 bp, Artificial Sequence
  oligonucleotide
acgagcattgcaccacaggagatcaacaccgcgtttacgcagaatttcttcagcgtgttt

SEQ ID NO: 26 - DNA, 60 bp, Artificial Sequence
  oligonucleotide
atcaaaaacttcaccctgctcgctaaacccaagtttgcggtagtagcccgaagccgacgt

SEQ ID NO: 27 - DNA, 60 bp, Artificial Sequence
  oligonucleotide
acctagggcgccttaggtgattcttttatacatcagaatgtgcggtcctaccggcggggt

SEQ ID NO: 28 - DNA, 38 bp, Artificial Sequence
  oligonucleotide
gaaagggcctatcaggccaattcgtcaacttcgtccac

SEQ ID NO: 29 - DNA, 35 bp, Artificial Sequence
  oligonucleotide
gctttggagctcttagatgagagatttcgattccg

SEQ ID NO: 30 - DNA, 47 bp, Artificial Sequence
  oligonucleotide
tatatatatcctaggtttttgtgatctgatgataagtggttggttcg

SEQ ID NO: 31 - DNA, 44 bp, Artificial Sequence
  oligonucleotide
atatatagaattccttcagcttagaaatgatgtgttttctacac

SEQ ID NO: 32 - DNA, 40 bp, Artificial Sequence
  oligonucleotide
atatatacaattgtagtacccttgtacttttggtttcacc

SEQ ID NO: 33 - DNA, 39 bp, Artificial Sequence
  oligonucleotide
tatatatgaattcggtttcgaggaagaagccacgaagtg

SEQ ID NO: 34 - DNA, 43 bp, Artificial Sequence
  oligonucleotide
atatataggatccggactgatgggctgcctgtatcgagtggtg

SEQ ID NO: 35 - DNA, 39 bp, Artificial Sequence
  oligonucleotide
tatatatgaattcagtacattaaaaacgtccgcaatgtg

SEQ ID NO: 36 - DNA, 41 bp, Artificial Sequence
  oligonucleotide
atatataggcgcgccatctcgtctctgttatgcttaagaag

SEQ ID NO: 37 - DNA, 60 bp, Artificial Sequence
  oligonucleotide
tatatatgggccctaataataatgtttaaacagaattagagctagaaatggaatggctgc

SEQ ID NO: 38 - DNA, 59 bp, Artificial Sequence
  oligonucleotide
gatccatggctagcaaaggagaagaacttttcactggagttgtcccaattcttgttgaa

SEQ ID NO: 39 - DNA, 60 bp, Artificial Sequence
  oligonucleotide
ttagatggtgatgttaatgggcacaaattttctgtcagtggagagggtgaaggtgatgct

SEQ ID NO: 40 - DNA, 60 bp, Artificial Sequence
  oligonucleotide
acatacggaaaacttacccttaaatttatttgcactactggaaaactacctgttccttgg

SEQ ID NO: 41 - DNA, 60 bp, Artificial Sequence
  oligonucleotide
ccaacacttgtcactactttctcttatggtgttcaatgcttttcccgttatccggatcag

SEQ ID NO: 42 - DNA, 60 bp, Artificial Sequence
  oligonucleotide
atgaaacggcatgactttttcaagagtgccatgcccgaaggttatgtacaggaacgcact

SEQ ID NO: 43 - DNA, 60 bp, Artificial Sequence
  oligonucleotide
atatctttcaaagatgacgggaactacaagacgcgtgctgaagtcaagtttgaaggtgat

SEQ ID NO: 44 - DNA, 60 bp, Artificial Sequence
  oligonucleotide
acccttgttaatcgtatcgagttgaaaggaatcgacttcaaggaagatggaaacatcctc

SEQ ID NO: 45 - DNA, 60 bp, Artificial Sequence
  oligonucleotide
ggacacaagctggagtacaactacaactcacacaacgtgtacatcaccgcagacaaacag

SEQ ID NO: 46 - DNA, 60 bp, Artificial Sequence
  oligonucleotide
aagaatggaatcaaagctaacttcaaaattcgccacaacattgaagatggttccgttcaa

SEQ ID NO: 47 - DNA, 60 bp, Artificial Sequence
  oligonucleotide
ctagcagaccattatcaacaaaatactccaattggtgatggccctgtccttttaccagac

SEQ ID NO: 48 - DNA, 60 bp, Artificial Sequence
  oligonucleotide
aaccattacctgtcgacacaatctgccctttcgaaagatcccaacgaaaagcgagaccac

SEQ ID NO: 49 - DNA, 60 bp, Artificial Sequence
  oligonucleotide
atggtccttcttgagtttgtaacagctgctgggattacacatggcatggatgagctctac

SEQ ID NO: 50 - DNA, 60 bp, Artificial Sequence
  oligonucleotide
aaatttgtgcccattaacatcaccatctaattcaacaagaattgggacaactccagtgaa

SEQ ID NO: 51 - DNA, 60 bp, Artificial Sequence
  oligonucleotide
aataaatttaagggtaagttttccgtatgtagcatcaccttcaccctctccactgacaga

SEQ ID NO: 52 - DNA, 60 bp, Artificial Sequence
  oligonucleotide
accataagagaaagtagtgacaagtgttggccaaggaacaggtagttttccagtagtgca

SEQ ID NO: 53 - DNA, 60 bp, Artificial Sequence
  oligonucleotide
ggcactcttgaaaaagtcatgccgtttcatctgatccggataacgggaaaagcattgaac

SEQ ID NO: 54 - DNA, 60 bp, Artificial Sequence
  oligonucleotide
cttgtagttcccgtcatctttgaaagatatagtgcgttcctgtacataaccttcgggcat

SEQ ID NO: 55 - DNA, 60 bp, Artificial Sequence
  oligonucleotide
tcctttcaactcgatacgattaacaagggtatcaccttcaaacttgacttcagcacgcgt

SEQ ID NO: 56 - DNA, 60 bp, Artificial Sequence
  oligonucleotide
tgagttgtagttgtactccagcttgtgtccgaggatgtttccatcttccttgaagtcgat

SEQ ID NO: 57 - DNA, 60 bp, Artificial Sequence
  oligonucleotide
aattttgaagttagctttgattccattcttctgtttgtctgcggtgatgtacacgttgtg

SEQ ID NO: 58 - DNA, 60 bp, Artificial Sequence
  oligonucleotide
tggagtattttgttgataatggtctgctagttgaacggaaccatcttcaatgttgtggcg

SEQ ID NO: 59 - DNA, 60 bp, Artificial Sequence
  oligonucleotide
aagggcagattgtgtcgacaggtaatggttgtctggtaaaaggacagggccatcaccaat

SEQ ID NO: 60 - DNA, 60 bp, Artificial Sequence
  oligonucleotide
agcagctgttacaaactcaagaaggaccatgtggtctcgcttttcgttgggatctttcga

SEQ ID NO: 61 - DNA, 55 bp, Artificial Sequence
  oligonucleotide
aaaaaaaaggcgcgcctcattatttgtagagctcatccatgccatgtgtaatccc

SEQ ID NO: 62 - DNA, 37 bp, Artificial Sequence
  oligonucleotide
gggatcccccaattcgtcaacttcgtccacagacatc

SEQ ID NO: 63 - DNA, 46 bp, Artificial Sequence
  oligonucleotide
tccaccatggatctgtcctgcagcccagcttagatgagagatttcg

SEQ ID NO: 64 - DNA, 43 bp, Artificial Sequence
  oligonucleotide
taaaaaaggtacctgtatgttattgtattgatctttcatgatg

SEQ ID NO: 65 - DNA, 48 bp, Artificial Sequence
  oligonucleotide
tttttttcctgcaggcatcgtaattttaagacgcagtctattatttcc

SEQ ID NO: 66 - DNA, 45 bp, Artificial Sequence
  oligonucleotide
atatatagcggccgcttcgctcgtgaaaagcagcgctcagggggc

SEQ ID NO: 67 - DNA, 39 bp, Artificial Sequence
  oligonucleotide
tatatatcctgcaggcggatagtaccgaggaaaggcagc

SEQ ID NO: 68 - DNA, 39 bp, Artificial Sequence
  oligonucleotide
atatatagtttaaacccgagaatgaacgccaagaggaac

SEQ ID NO: 69 - DNA, 48 bp, Artificial Sequence
  oligonucleotide
tatatatatgggcccggccgacgcgaagcggcggggcgtagggagcgc

SEQ ID NO: 70 - DNA, 57 bp, Artificial Sequence
  oligonucleotide
aattcatttacaattgaatatatcctgccattttacacctcgatatatcctgccaca

SEQ ID NO: 71 - DNA, 57 bp, Artificial Sequence
  oligonucleotide
aatttgtggcaggatatatcgaggtgtaaaatggcaggatatattcaattgtaaatg

SEQ ID NO: 72 - DNA, 40 bp, Artificial Sequence
  oligonucleotide
atatatagggccccgctcaccgggctggttgccctcgccg

SEQ ID NO: 73 - DNA, 42 bp, Artificial Sequence
  oligonucleotide
atatatattgtacagtaaagcgctggctgaacccccagccgg

SEQ ID NO: 74 - DNA, 36 bp, Artificial Sequence
  oligonucleotide
gatcggtaccgttcctgcaggaaccatggttcggcc

SEQ ID NO: 75 - DNA, 28 bp, Artificial Sequence
  oligonucleotide
gaaccatggttcctgcaggaacggtacc